(19) United States
(12) Patent Application Publication
(10) Pub. No.: US 2024/0122499 A1
(43) Pub. Date: Apr. 18, 2024

(54) WEARABLE INERTIAL SENSOR SYSTEM AND METHODS

(71) Applicant: NEW YORK UNIVERSITY, New York, NY (US)

(72) Inventors: Ashwin Rajkumar, Brooklyn, NY (US); Fabio Vulpi, Bari (IT); Preeti Raghavan, Brooklyn, NY (US); Vikram Kapila, West Orange, NJ (US)

(21) Appl. No.: 18/257,609

(22) PCT Filed: Dec. 16, 2021
(86) PCT No.: PCT/US2021/063760

§ 371 Date: Jun. 15, 2023

Related U.S. Application Data
(60) Provisional application No. 63/126,216, filed on Dec. 16, 2020.

(51) Int. Cl. A61B 5/11; A61B 5/00; A61B 5/107; A63F 13/211; A63F 13/212; G16H 40/40; G16H 40/67

(52) U.S. Cl. CPC A61B 5/1114; A61B 5/1071; A61B 5/6823; A61B 5/6824; A61B 5/7475; A63F 13/211; A63F 13/212; G16H 40/40; G16H 40/67; A61B 2560/0223; A61B 2562/0219

(57) ABSTRACT

Disclosed is a wearable inertial sensors (WIS) system and methods for real-time simultaneous tri-planar motion capture of the upper extremity (UE). The sensors simultaneously capture in the frontal, sagittal, and horizontal planes UE range of motion (ROM), which is critical to assess an individual's movement limitations and determine appropriate rehabilitative treatments. Off-the-shelf sensors and microcontrollers are used to develop the WIS system, which wirelessly streams real-time joint orientation for UE ROM measurement. Key developments include: (i) two novel approaches, using earth's gravity (EG approach) and magnetic field (EGM approach) as references, to correct misalignments in the orientation between the sensor and its housing to minimize measurement errors; (ii) implementation of the joint coordinate system (JCS)-based method for tri-planar ROM measurements for clinical use; and (iii) an in-situ guided mounting technique for accurate sensor placement and alignment on human body.

### CROSS-REFERENCE TO RELATED APPLICATIONS

This application claims priority to U.S. provisional application No. 63/126,216 filed on Dec. 16, 2020, incorporated herein by reference in its entirety.

### STATEMENT REGARDING FEDERALLY SPONSORED RESEARCH OR DEVELOPMENT

This invention was made with government support under grant number P2CHD086841 awarded by the National Institutes of Health. The government has certain rights in the invention.

### BACKGROUND

Recent years have witnessed the emergence of digital health using advanced technologies such as wearable sensors and embedded controllers to enhance access to medical diagnostics and treatments. Because of an accelerating trend in the number of stroke survivors requiring rehabilitation, healthcare services worldwide are considering technological solutions to enhance accessibility to assessment and treatment. For example, virtual therapists and telerehabilitation have been proposed to complement the skills of therapists. However, some of the challenges faced by these technologies are a lack of clinical acceptance, high equipment costs, reduced accuracy, and a lack of user friendliness in a clinical and home setting.

Most tasks constituting activities of daily living have minimum range of motion (ROM) requirements at various joints. Restoring the ability to perform activities of daily living in individuals with impaired movement therefore requires clinicians to assess ROM and customize the exercises to each patients' activity limitations. Commercially available devices such as goniometers, inclinometers, and videographic methods are used by therapists to assess patient's ROM in one-on-one clinical settings. Goniometers and inclinometers have limited inter-observer agreement due to variability in positioning the sensors on the patient's body and can capture motion only for one joint at a time. Videographic methods also show low inter-observer agreement due to differences in camera positions and can provide only two-dimensional information of three-dimensional motion, which is particularly problematic when movements are abnormal. Hence, these methods are not suitable for remote assessments and individualized treatments, which are essential to enhance accessibility.

One approach for ROM and rehabilitation assessment applications is to use markerless motion capture system used for gaming, such as the Kinect™ V2 (Microsoft Corp., Redmond, WA). The Kinect can provide high-definition video output, depth information, and position information for 25 joints of a human in 3D space. Several studies have reported on the use of the Kinect sensor for ROM assessment and rehabilitative applications. Use of the Kinect for gait analysis and joint angle orientation measurements has shown varying levels of agreement for different joint segments. Under ideal conditions, the shoulder and elbow joint ROM measurements from the Kinect show good inter-trial repeatability and correlate with measurements taken with a goniometer. However, the placement of Kinect is a limiting factor: placing it in front of a subject yields higher reliability measurements in contrast to placing it on the side, but even then fails to correctly measure elbow flexion-extension from a neutral position and cannot measure forearm pronation-supination. Furthermore, a recent literature review on the use of repurposed gaming consoles, including Kinect, for neurorehabilitation in several target populations reported an inability to provide individualized training as a major limitation of the current systems.

An alternative approach for ROM and rehabilitation assessment applications is to use inertial sensors, which include inertial measurement units (IMU) and magnetic, angular rate, and gravity (MARG) sensors that measure the linear acceleration and angular velocity of a rigid body to which they are attached. Commercially available wearable inertial sensors for motion capture can be used for ROM assessment, but their use for rehabilitation is limited by the cost of custom data acquisition software, need for user-training, and the extensive data analysis required post-acquisition. Although studies have shown that it is possible to extract absolute orientation of a rigid body from raw measurements of the IMU and MARG sensors, their methods are yet to be extended for use in rehabilitation applications requiring joint angle measurements.

Telerehabilitation is a branch of emerging medical innovation that permits assessment and treatment of patients remotely. Repurposed off-the-shelf media platforms (e.g., Skype, VSee, etc.) and interactive gaming consoles (e.g., Kinect, PlayStation, Wii, etc.) have been used in prior research efforts for telerehabilitation. However, most telerehabilitation applications are limited by (i) an inability to capture measurements accurately and with high inter-observer agreement easily in a remote manner and (ii) provide individualized coaching asynchronously, i.e. without a live coach.

### SUMMARY

Some embodiments of the invention disclosed herein are set forth below, and any combination of these embodiments (or portions thereof) may be made to define another embodiment.

In one aspect the present invention relates to a wearable inertial sensor system to detect upper extremity movement comprising a plurality of inertial sensors configured to removably connect to a plurality of mounting devices, and a computing system wirelessly and communicatively connected to the plurality of inertial sensors, further configured to provide a plurality of user interfaces for rehabilitative exergames.

In one embodiment, a plurality of joint angles are calculated in a joint angle coordinate system by the computing system based on quaternion data provided by the plurality of inertial sensors. In one embodiment, the joint angels are calculated in real time and shown in the exergame user interface. In one embodiment, one of the plurality of inertial sensors is configured to be placed on a subject's left forearm. In one embodiment, one of the plurality of inertial sensors is configured to be placed on a subject's left upper arm. In one embodiment, one of the plurality of inertial sensors is configured to be placed on a subject's right forearm. In one embodiment, one of the plurality of inertial sensors is configured to be placed on a subject's right upper arm. In one embodiment, one of the plurality of inertial sensors is configured to be placed centrally on a subject's back. In one embodiment, the system further comprises a calibration device.

In one embodiment, one of the plurality of user interfaces is a sensor calibration user interface. In one embodiment, one of the plurality of user interfaces is a sensor mounting user interface. In one embodiment, one of the plurality of user interfaces is a patient user interface. In one embodiment, one of the plurality of user interfaces is a playback user interface. In one embodiment, one of the plurality of user interfaces is an instructor user interface.

In one embodiment, the system further comprises a non-transitory computer-readable medium with instructions stored thereon, that when executed by a processor, performs the steps of calibrating the plurality of inertial sensors, correcting for gravity based misalignment, correcting for magnetic field based misalignment, collecting inertial sensor data, calculating relative quaternion positions between the inertial sensors, converting the calculated quaternion positions to joint angles, and displaying the joint angles to a user via a user interface. In one embodiment, the joint angles are displayed to a user via the user interface in real-time.

In another aspect a joint angle calculation method comprises calibrating a plurality of inertial sensors, correcting for gravity based misalignment, correcting for magnetic field based misalignment, collecting inertial sensor data, calculating relative quaternion positions between the inertial sensors, converting the calculated quaternion positions to joint angles, and displaying the joint angles to a user via a user interface.

In another aspect a non-transitory computer-readable medium for calculating joint angles for exergames comprises a computer program code segment used to communicate with a plurality of inertial sensors, a computer program code segment used to calibrate the plurality of inertial sensors, a computer program code segment used to collect inertial sensor data, a computer program code segment used to calculate relative quaternion positions between the inertial sensors, a computer program code segment used to convert the calculated quaternion positions to joint angles, and a computer program code segment used to present the joint angles on a exergame user interface.

In one embodiment, the computer-readable medium includes instructions stored thereon, that when executed by a processor, performs the steps of calibrating the plurality of inertial sensors, correcting for gravity based misalignment, correcting for magnetic field based misalignment, collecting inertial sensor data, calculating relative quaternion positions between the inertial sensors, converting the calculated quaternion positions to joint angles, and displaying the joint angles to a user via a user interface.

In another aspect, the present invention relates to a method of accurately placing inertial sensors on a subject, comprising the steps of placing a first inertial sensor on an upper body of the subject; placing a second inertial sensor on one or both of the subject's left and right upper arms; and placing a third inertial sensor on one or both of the subject's left and right forearms; wherein a carrying angle of one or both of the subject's left and right arms is between about 8° and 20°, and wherein an internal-external rotation of one or both of the subject's left and right shoulder joints is within about 5° of a neutral pose.

In one embodiment, the first inertial sensor is positioned on a lower back of the subject. In one embodiment, the second inertial sensor is positioned just proximal to one or both of the subject's elbow joint. In one embodiment, the third inertial sensor is positioned just proximal to one or both of the subject's wrist joint.

In another aspect, the present invention relates to a method of diagnosing an upper body mobility disease or disorder in a subject, comprising the steps of instructing a specific movement and measuring a range of motion (ROM) for instructed as well as non-instructed movements simultaneously, quantifying primary and secondary movement restrictions and compensatory strategies in real-time using computer algorithms at baseline, characterizing one or more diseases or disorders in the subject based on the quantifications, and tracking movements after one or more treatments.

In one embodiment, the joint is selected from the group consisting of: the shoulder, the elbow, and the wrist. In one embodiment, the joint ROM is selected from the group consisting of: shoulder flexion, shoulder extension, shoulder abduction, shoulder adduction, shoulder internal rotation, shoulder external rotation, shoulder protraction, shoulder retraction, shoulder plane, shoulder elevation, elbow flexion, elbow extension, carrying angle, wrist pronation, wrist supination, wrist radial deviation, wrist ulnar deviation, wrist palmarflexion, wrist dorsiflexion, finger flexion, finger extension, finger abduction, finger adduction, thumb flexion, thumb extension, thumb opposition, thumb abduction, thumb adduction. The embodiment can be extended to include neck flexion, neck extension, neck rotation, next lateral bending, spine flexion, spine extension, spine lateral bending, spine rotation, hip flexion, hip extension, knee flexion, knee extension, ankle plantar flexion, ankle dorsiflexion, eversion, inversion, toe flexion, and toe extension. In one embodiment, the disease or disorder is selected from the group consisting of: stroke, multiple sclerosis, spinal cord injury, nerve damage, rheumatism, arthritis, fracture, sprain, stiffness, weakness, impaired coordination, impaired proprioception, epicondylitis, tendonitis, and hypermobility.

In one embodiment, the deviation is an increase or decrease in joint ROM of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In one embodiment, movement impairment is detected in joints having movement in a sagittal plane, a frontal plane, and/or a horizontal plane. In one embodiment, the baseline joint ROM is derived from a population selected from the group consisting of: a global population, a regional group, an ethnic group, an age group, a gender group, a healthy subject, a subject having a disorder or disease, a subject at a stage of progression of a disorder or disease, and a subject at a stage of treatment of a disorder or disease. In one embodiment, joint ROM data predicts progression of a disease or condition. In one embodiment, a derived joint ROM is used to predict a progression of a disease or disorder and a therapeutic intervention based on characteristic patterns of ROM deviation from baseline specific to the disease or disorder. In one embodiment, the method further comprises a step of administering a treatment and a step of measuring changes in joint ROM after treatment.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 shows a wearable inertial sensor system in accordance with some embodiments.

FIG. 2 shows further details of the system in accordance with some embodiments.

FIG. 3 shows features of the sensors of the system in accordance with some embodiments.

FIG. 4 shows a schematic representation of a printed circuit board (PCB) of the system in accordance with some embodiments.

FIG. 5 shows a 3D printed housing for the PCB in accordance with some embodiments.

FIG. 6 shows a calibration holder for the system in accordance with some embodiments.

FIG. 7 shows an example user interface (UI) in accordance with some embodiments.

FIG. 8 shows an example UI in accordance with some embodiments.

FIG. 9 shows an example UI in accordance with some embodiments.

FIG. 10 shows an example UI in accordance with some embodiments.

FIG. 11 shows an example UI in accordance with some embodiments.

FIG. 12 shows a schematic representation of a computer of the system in accordance with some embodiments.

FIG. 13 shows a table of example calibration and mounting times in accordance with some embodiments.

FIG. 14 shows an image of a calibration experiment for the system in accordance with some embodiments.

FIG. 15 shows example coordinate systems of the system in accordance with some embodiments.

FIG. 16 is a flow chart showing a method of orientation misalignment correction in accordance with some embodiments.

FIG. 17 shows a table of example module angles relative to gravity in accordance with some embodiments.

FIG. 18 shows a calibrated experimental platform in accordance with some embodiments.

FIG. 19 is a flow chart showing a correction method in accordance with some embodiments.

FIG. 20 shows a table of example experimental angular rotation results in accordance with some embodiments.

FIG. 21 shows an example experimental goniometer in accordance with some embodiments.

FIG. 22 shows an example experimental UI in accordance with some embodiments.

FIG. 23 shows a table of example experimental computation times in accordance with some embodiments.

FIG. 24 shows a table of example experimental comparison data in accordance with some embodiments.

FIG. 25 shows a table of example experimental measured angles in accordance with some embodiments.

FIG. 26 shows example experimental data in accordance with some embodiments.

FIG. 27 shows example experimental data in accordance with some embodiments.

FIG. 28 shows example joint positions, joint vectors and anatomical planes in accordance with some embodiments.

FIG. 29 shows an example experimental setup in accordance with some embodiments.

FIG. 30 shows example comparative experimental data in accordance with some embodiments.

FIG. 31 shows a table of example comparative experimental data in accordance with some embodiments.

FIG. 32 shows example comparative experimental data in accordance with some embodiments.

FIG. 33 shows example comparative experimental data in accordance with some embodiments.

FIG. 34 shows a table of dominant joint ROM data from 10 male subjects, mean (standard deviation).

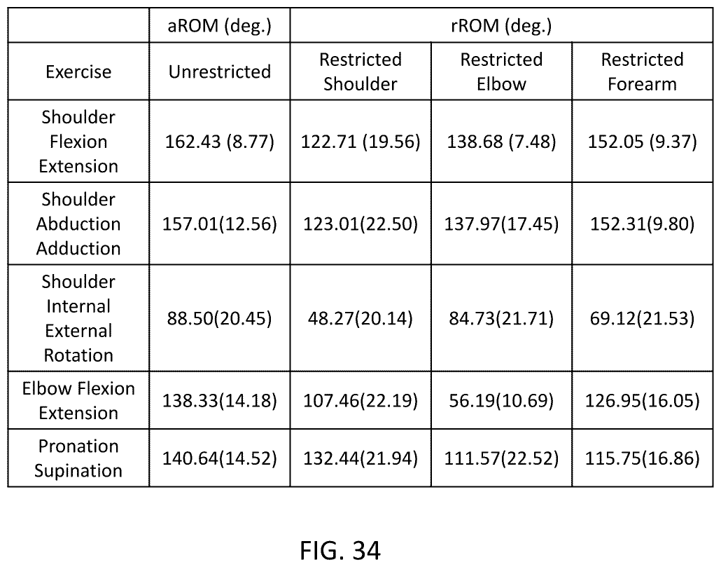

FIG. 35 shows a table (top) and graph (bottom) of percentage reduction in joint ROM after restriction compared to the data from FIG. 34.

FIG. 36 shows a table of dominant joint ROM data from 10 female subjects, mean (standard deviation).

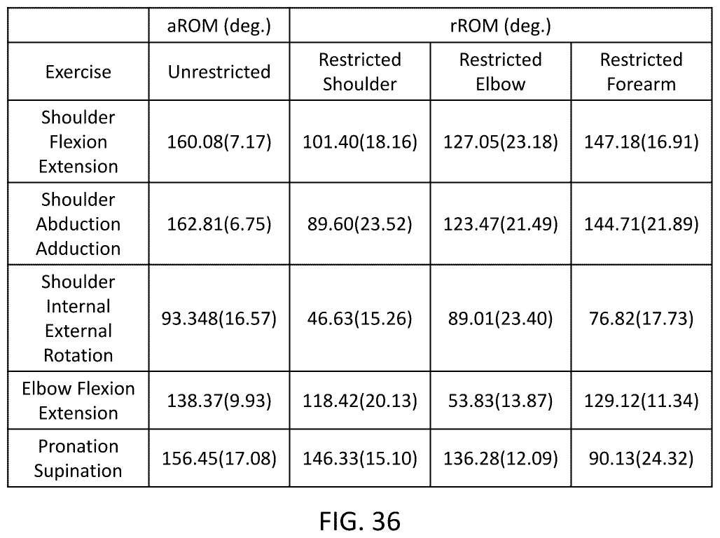

FIG. 37 shows a table (top) and graph (bottom) of percentage reduction in joint ROM after restriction compared to the data from FIG. 36.

FIG. 38 shows a table of dominant joint ROM data from the 10 male and 10 female subjects from FIG. 34 and FIG. 36, mean (standard deviation).

FIG. 39 shows a table (top) and graph (bottom) of percentage reduction in joint ROM after restriction compared to the data from FIG. 38.

FIG. 40 shows tables of unrestricted joint ROM data and restricted joint ROM data from male subject number 3, mean (standard deviation).

FIG. 41 shows tables of unrestricted joint ROM data and restricted joint ROM data from male subject number 6, mean (standard deviation).

FIG. 42 shows tables of unrestricted joint ROM data and restricted joint ROM data from female subject number 1, mean (standard deviation).

FIG. 43 shows tables of unrestricted joint ROM data and restricted joint ROM data from female subject number 4, mean (standard deviation).

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of wearable inertial sensors. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a wearable inertial sensor system and methods.

To address of limitations current technologies for ROM and rehabilitation assessment applications, disclosed is a wearable inertial sensors (WIS) for exergames (WISE) system. In certain embodiments, the system and method of the present invention relate to assessing, tracking, and monitoring of a subject's upper extremity movement. In some embodiments, the system is configured to identify weakness, stiffness, joint restrictions, coordination problems, and/or impaired sensation, and can further be configured to track improvement of these issues during treatment. In some embodiments, the system is configured to diagnose joint restrictions at a first joint that is affecting a second joint. For example, in certain aspects the system and method are used to detect or diagnose the cause of improper movement, which in certain instances can lead to the development of rational treatment plans. For example, in certain aspects, the system and method can be used to monitor movement related to shoulder external rotation, shoulder abduction, shoulder flexion, elbow extension, forearm supination, and/or finger extension. In one embodiment, the system and method can detect restrictions in one or more of these movements. In certain aspects, the system and method are used to train a subject for proper movement. For example, in one embodiment, the system and method enables training of movements related to shoulder external rotation, shoulder abduction, shoulder flexion, elbow extension, forearm supination, and/or finger extension. In one embodiment, the training of such movements enable the recovery of function. In one embodiment, the system includes an animated virtual coach to deliver virtual instruction for any activity, and a subject-model whose movements are animated by real-time sensor measurements using inertial sensors worn by a subject.

Arm movements are crucial for performing several activities of daily living (ADL). Each ADL has minimum range of motion (ROM) requirements for the upper extremity (UE) joints to successfully complete the task. However, neurological events such as stroke, multiple sclerosis, spinal cord injury, nerve damage, etc., can limit an individual's ROM, which in turn prevents them from performing several ADLs and lowers their quality of life. The pathway for recovery of lost motor skills includes: (i) determining the movement limitations to facilitate development of a treatment plan and (ii) gauging recovery to tailor treatment changes based on patient progress. The anatomy of the human arm with seven degrees of freedom requires advanced motion capture systems to measure the complex movements at the shoulder, elbow, and wrist.

Existing tools for ROM assessment used in clinical practice include: (i) hand-held measurement devices such as a goniometer, inclinometer, etc.; and (ii) video analysis software such as the Dartfish. However, these devices do not capture all the degrees of freedom of arm motion. In a research setting, several commercially available motion capture devices are used that can be broadly classified under: (i) marker-based optical motion capture systems; (ii) electromagnetic position tracking systems; (iii) marker-less motion capture systems; and (iv) inertial sensing systems. However, these systems require trained personnel for setup and data processing, which prohibits their translation from research to clinical practice and home-based environments.

Moreover, providing healthcare rehabilitative services for the aging baby boomer population requires tech-savvy solutions to augment the therapists and clinicians for effective remote monitoring and telemedicine. Video and computer gaming facilitate an entertaining and engaging user experience while performing monotonous repetitive exercises and improve the therapeutic benefits of the treatment.

In one embodiment, kinematic assessment performed by the WISE system is used to identify the key movement abnormalities. Detection of the cause of the abnormalities, such as weakness vs stiffness, is also performed. This enables a correction of the movement abnormalities through targeted exercises guided by a virtual coach.

Disclosed are details of the salient features of an exergame (exercise combined with game) that extends prior work in developing a wearable inertial sensors (WIS) system. The International Society of Biomechanics has recommended the use of the joint coordinate system (JCS) for comprehensive visualization of the tri-planar limb movements. As described herein, the present invention relates to the development of a mechatronics based WIS system that utilizes JCS for ROM assessment.

MOCAP is an interdisciplinary research topic that focuses on quantifying motion and enabling interaction in real and virtual environments. Commercially available MOCAP systems can be broadly classified into: (i) optical marker-based systems, (ii) electromagnetic position tracking system, (iii) markerless optical systems, and (iv) inertial sensing systems. Marker-based optical MOCAP is the gold standard for tracking joint position and angular movement with high precision and accuracy. Nevertheless, such systems require precise marker placement and expensive cameras, all of which are burdensome for clinical use. Furthermore, marker occlusion can occur during limb movements, making tracking difficult. Electromagnetic position tracking (e.g., by Ascension Technology Corp.) computes the position of body-worn electromagnetic sensors relative to a transmitter. These systems avoid the use of multiple cameras and marker occlusion, but they are not easy to use for clinical purposes. Markerless optical systems such as the Kinect™ V2 (Microsoft Corp., Redmond, WA) is a popular MOCAP device to measure joint positions in 3D space. However, data from the Kinect and other markerless video-analysis systems cannot make measurements in the horizontal plane, such as shoulder internal-external rotation and forearm pronation-supination, which are critical for ADL. Furthermore, the Kinect cannot be used in noisy visual environments. Recent advancements in deep learning with markerless MOCAP using videography can reduce the human effort to track human and animal behavior, but these have the same limitations as other vision-based systems such as the Kinect, and do not provide precise tri-planar measurements for real-time applications.

Inertial sensors refer to a family of sensors capable of measuring the pose of a rigid body in 3D space. Commercially available inertial sensors for MOCAP (e.g. Opal, X-sens, etc.) are expensive due to their built-in calibration techniques, sensor fusion algorithms, offset correction techniques, and software support, and are not suitable for translation to at-home use and clinical practice.

Sensor fusion algorithms for extracting orientation information from the inertial sensors' raw accelerometer, gyroscope, and magnetometer data have been developed. These fusion algorithms have been considered in the context of a single inertial sensor but are yet to be explored in MOCAP applications that require simultaneous use in a network of multiple inertial sensors. Consumer targeted inertial sensors can be used for numerous medical diagnostic applications as discussed in. However, for single joint motion, the approach does not produce clinically usable tri-planar measurements. A comparison of commercial MOCAP systems vs. consumer-grade inertial sensors for UE ROM measurements has been previously performed and the results suggest that consumer-grade sensors can provide similar accuracy as commercial MOCAP systems. Finally, a state-of-the-art review on using inertial sensors for MOCAP recommends the use of JCS-based ROM reporting as human motion includes tri-planar movements requiring simultaneous measurements in multiple axes.

Inertial sensors require an initial calibration and correction for various misalignment errors to provide accurate measurements. Specifically, obtaining precise measurements from low-cost inertial sensors requires the following steps: (i) calibration of individual sensors of the IMU or MARG system; (ii) correction of misalignment arising from the offset between the inertial sensor and the housing containing it; and (iii) anatomical calibration required due to misalignment of inertial sensor with the object that it is being mounted on. To retrieve accurate measurements from individual sensors (i.e., accelerometer, gyroscopes, and magnetometer), the calibration procedure corrects for the errors arising from: (i) scaling factors, (ii) cross axis sensitivity, (iii) offsets in the three axes (non-orthogonality), etc. Comprehensive approaches for calibration of IMU sensors utilizing sensor error models for accelerometers and gyroscopes are presented in previous work. Dynamic model-based adaptive control techniques to improve the performance of micro-gyroscopes are presented in previous work. Nonetheless, recent years have witnessed inertial sensor packages endowed with on-board microcontrollers to support self-calibration. For example, the BNO055 device offers simple experimental routines for calibration that can be performed by novice users, effectively obviating the need for individual sensor calibration techniques.

Sensor-housing offset arises when the sensor orientation does not align with the orientation of sensor housing. For such a case, effective orientation misalignment correction techniques need to be developed to reprocess the sensor measurement and align it with the housing to obtain accurate measurement. A rotation matrix-based orientation misalignment correction method using a calibration device was developed, which yields unique results for a specific sensor and its housing and needs to be repeated for each sensor/housing pair. Alternatively, the system utilizes a simpler and computationally efficient quaternion-based approach to develop two orientation misalignment correction methods for the inertial sensors.

Finally, anatomical calibration is essential for accurate measurement of joint angles from the human body. Alignment free calibration of wearable inertial sensors for the human body has been examined by using prescribed motion sequences in the upper and lower extremities. However, a limitation of such an approach is that the individual must initiate movements from a standard position, which may not be achievable for persons with movement limitations.

Disclosed is a WIS system 100 for UE ROM assessment using inertial sensors that wirelessly stream quaternion data for the absolute orientation of the sensors. In one embodiment, two sensor-to-housing orientation misalignment correction techniques were developed to use the quaternion measurements from the inertial sensors and retrieve absolute orientation of the housing. The JCS approach is utilized to compute the ROM data from the quaternion measurements obtained from the sensors worn by the subject. An in-situ data-driven technique for mounting and aligning the WIS on the human body is used for precise placement of the sensors, resulting in accurate measurement of ROM.

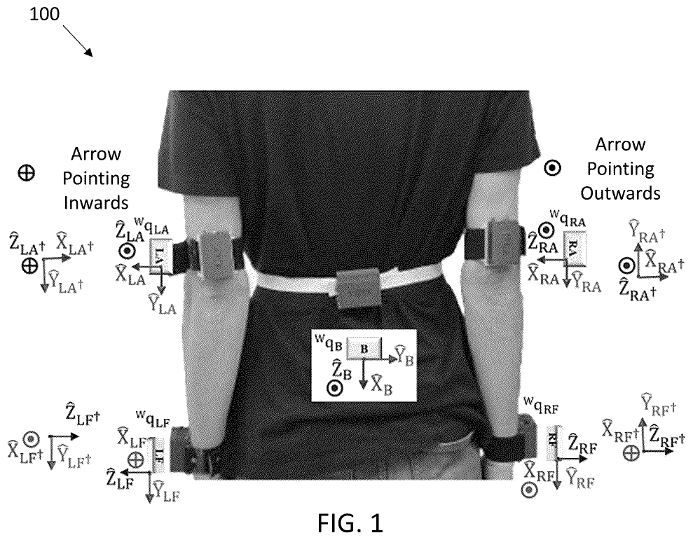

FIG. 1 shows a wearable inertial sensor system (WIS, WISE) 100 including a plurality of inertial sensor modules. Additionally, FIG. 1 shows coordinate frames and WISE module axes references for computation of shoulder and elbow joint angle orientations.

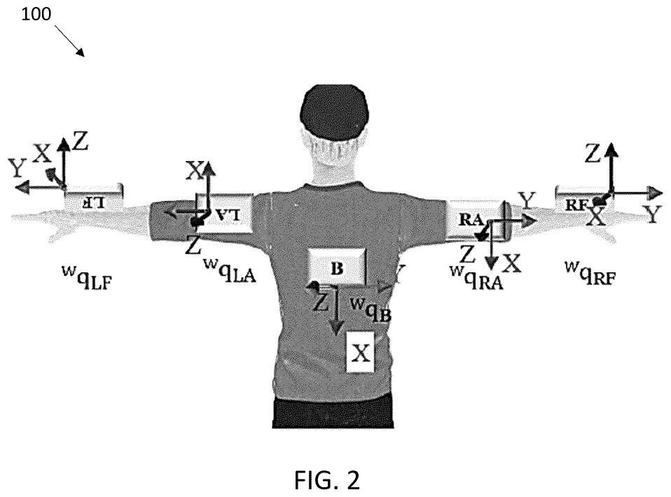

FIG. 1 shows a human subject wearing the WIS system in neutral pose while FIG. 2 shows a WIS module pictorially represented on a human model in T-pose. The WIS modules, each fitted with an inertial sensor (such as a BNO055 sensor), require an initial calibration of their built-in tri-axial magnetometer, accelerometer, and gyroscopes (MARG) sensors for accurate absolute orientation measurement. Furthermore, precise mounting on the human body is crucial for accurate measurement of ROM.

Individuals with movement limitations may have highly variable initial positions. Hence to be truly applicable in a clinical setting, the sensing method should be able to measure joint ROM accurately from any initial position of the extremity. The present system was thus developed to (i) measure the initial pose of the arm from absolute orientation of the body-worn sensors and (ii) measure the ROM simultaneously in the three planes at the shoulder and elbow. To achieve these objectives, individual sensors worn on each body segment must sense the orientation relative to the previous body segment. Prior work shows that mounting the sensors at the distal end of arm segments from the joints whose motion is being measured increases the accuracy of joint angle measurement. Moreover, wireless connectivity such as the Bluetooth low energy (BLE) protocol can eliminate the hassle of being tethered to a computer. In one embodiment, the WIS system, for example, can include five wireless inertial sensors mounted on the body as shown in FIG. 1 and FIG. 2, where LF, RF, LA, and RA represent sensors mounted on the left and right forearm and left and right arm, respectively, and B refers to the sensor mounted on the back. In one embodiment, the sensors are mounted using straps and a belt. However, the present invention is not limited to any particular number of inertial sensors or placement of said sensors. Rather, the present invention encompasses systems comprising any number of inertial sensors that can accurately measure UE ROM.

In one embodiment, the sensor modules are integrated with microcontrollers to stream their absolute orientation quaternions relative to the earth's magnetic and gravitation fields. In one embodiment, the microcontrollers stream their data using Bluetooth, Wi-Fi, RFID, or any other known technology capable of data transmission. In one embodiment, the absolute quaternions are further converted to relative quaternions to obtain the joint angles of each arm segment. Furthermore, the results of a pre-clinical study on testing the usability and accuracy of the WIS system in contrast to an alternative motion capture technology is presented below.

In one embodiment, the WIS system 100 employs off-the-shelf inertial sensors and microcontrollers to facilitate translation of technology to clinical settings. The multi-module wearable sensor framework of this disclosure requires the use of a star topology that enables wireless connectivity of multiple devices to a single host computer, tablet or smartphone interface. The Gazell protocol from Nordic semiconductors is a common peer-to-peer star topology network. The RFduino microcontroller that supports BLE and Gazell protocols was chosen for the design of the WIS system.

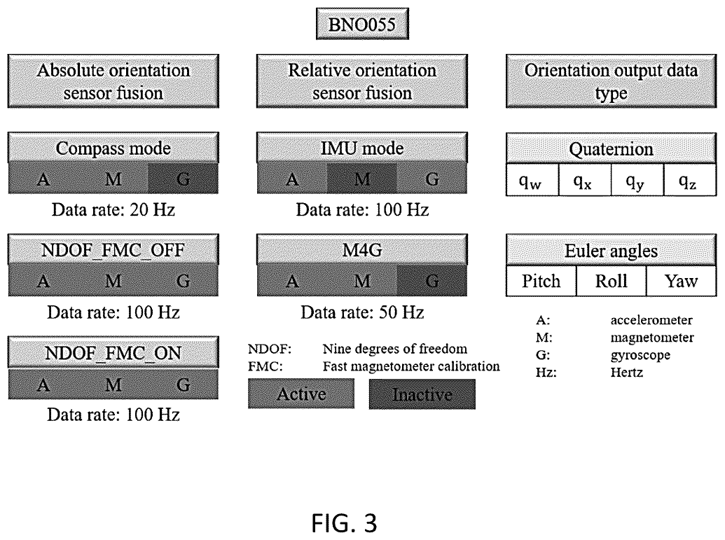

Several low cost, consumer targeted MARG sensors, e.g., BNO055, MPU9150, and X-NEUCLEO can serve as inertial sensors in the proposed WIS system. While these three sensors can provide absolute orientation, the BNO055 has superior static and dynamic angular measurement stability over the MPU9150 and X-NEUCLEO. Moreover, the BNO055′s direct sensor fusion and various operating modes were deemed to offer a high degree of flexibility for the WIS system over the MPU9150. Specifically, the BNO055 sensor can measure (i) absolute orientation relative to the earth's magnetic field and gravity and (ii) relative orientation from its initial start position based on the selected operating mode. The absolute orientation signals from the sensor can be retrieved using the following operating modes (i) compass mode, (ii) nine degrees of freedom fast magnetometer calibration off mode (NDOF_FMC_OFF), and (iii) nine degrees of freedom fast magnetometer calibration on mode (NDOF_FMC_ON). The NDOF modes require an initial calibration of the three sensors (three axis accelerometer, magnetometer, and gyroscope) for streaming absolute orientation. In any operating mode, the absolute or relative orientation output data from BNO055 is obtainable as quaternions or Euler angles. Salient features of the BNO055 sensor are delineated in FIG. 3.

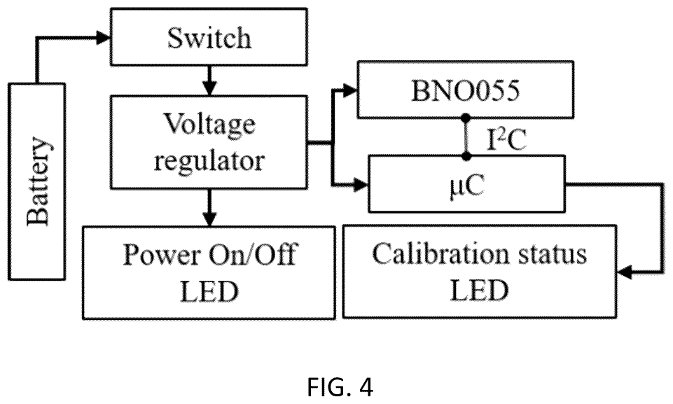

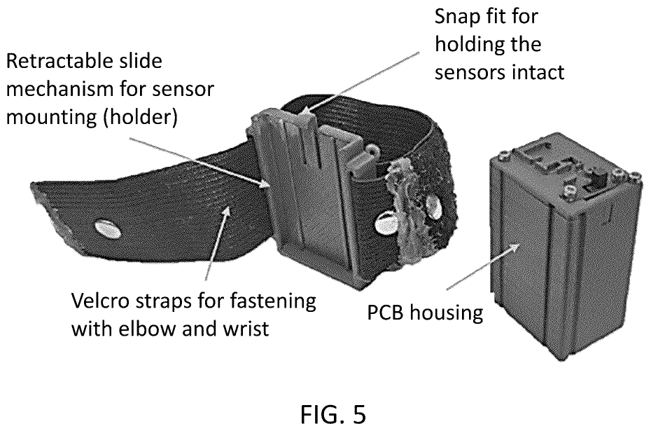

In one embodiment, the wearable inertial sensors wirelessly connect to a computing device (e.g., computer, tablet, smartphone, etc.) to stream the quaternion data corresponding to each sensor's absolute orientation. In one embodiment, the sensors wirelessly connect to a USB host tethered to the computing device. In one embodiment, system 100 comprises wearable housings designed for hosting the sensors and mounting them to the human body. In one embodiment, the wearable housings are 3D printed. FIG. 4 shows the schematic representation of the printed circuit board (PCB) developed and FIG. 5 shows the 3D printed housing for the PCB. In one embodiment, the quaternion data for absolute orientation is obtained from each sensor through I2C communication and a packet of maximum 18 bytes, containing each sensor's quaternion and its corresponding identifier, is created for streaming through the Gazell protocol to the computer.

Euler angle, quaternion, and axis/angle representations are the commonly used methods to describe the absolute orientation of a rigid body. Tait-Bryan angles, a subset of Euler angles, utilize three angles about the axes of the world coordinate frame to describe the rotation of the body. However, utilizing Euler angles to describe rigid body rotations often results in gimbal lock and singularity problems. Moreover, the computational simplicity of quaternions (four elements) vs. rotation matrices (six elements) suggests the use of quaternions for rotation description. A brief review of quaternions is included below for completeness.

A quaternion is a four-tuple representation of the orientation of a coordinate frame in 3D space. A quaternion describing the rotation of a coordinate frame given by the axis/angle representation (k, ϕ), where k=[kₓ k_{y} k_{z}]^{T}, is characterized below.

$${q}_{k} ( ϕ ) := ( \begin{matrix}cos \frac{ϕ}{2} & {k}_{x} sin \frac{ϕ}{2} & {k}_{y} sin \frac{ϕ}{2} & {k}_{z} sin \frac{ϕ}{2}\end{matrix} ) \qquad ( 1 )$$

A quaternion q:=(q_{w} qₓ q_{y} q_{z}) includes a scalar q_{w} and a vector [symbol] :=[qₓ q_{y} q_{z}]. Throughout this paper, vectors are denoted using uppercase alphabets, such as Q, and quaternions using lowercase alphabets, such as q. Moreover, “\hat{ }” and “˜” are used for vectors and quaternions represented in the world frame and sensor frame, respectively. Consistent with the notation in prior literature, “⊗” and “*” are used to denote quaternion product and conjugate in this paper.

In some embodiments, the system 100 further comprises a non-transitory computer-readable medium with instructions stored thereon, that when executed by a processor, performs the steps of calibrating the plurality of inertial sensors, correcting for gravity based misalignment, correcting for magnetic field based misalignment, collecting inertial sensor data, calculating relative quaternion positions between the inertial sensors, converting the calculated quaternion positions to joint angles, and displaying the joint angles to a user via a user interface. In some embodiments, the joint angles are displayed to a user via the user interface in real-time.

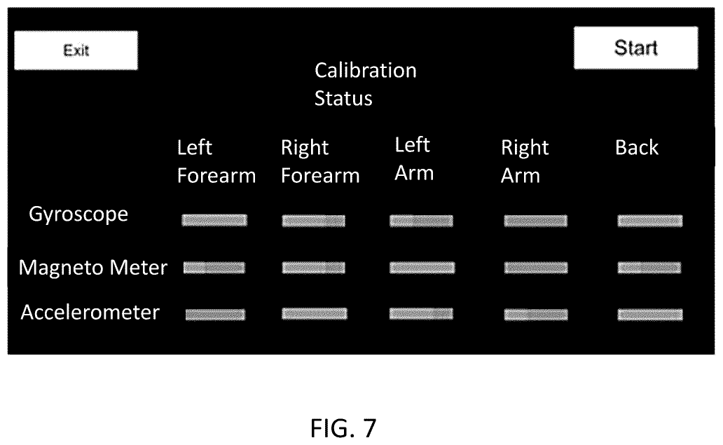

FIGS. 7, 8, 9, 10 and 11 show examples of a user interface (UI) for utilizing the system 100. A prior experimental study leveraged MATLAB-based user interfaces (UIs) for data acquisition, calibration, and in-situ sensor mounting on the subject's body. The MATLAB-based Uls were best suited for experimentation with researchers, but less suitable for use by patients and therapists. Moreover, another prior study found that the use of an animated virtual coach for ROM training improves system usability. One design motivation for the exergame is to develop a holistic application that integrates multiple interfaces for sensor calibration, sensor mounting, and sensor data collection to assist patients and clinicians. Furthermore, such data-driven rehabilitative approaches will enable clinicians to tailor personalized exercises to patients' needs paving a pathway for precision rehabilitative treatment.

In one embodiment, an exergame environment has been developed using the Unity3D software application to retrieve the data from the WIS modules and display the same with unique interfaces: (i) calibration UI for visualization and assistance with sensor calibration; (ii) sensor mounting UI for guided mounting of WIS modules on the human body; (iii) patient UI for practicing ROM exercises; (iv) playback UI for visualization of patient's performance by clinicians; and (v) instructor UI for creation of customized exercises for patients.

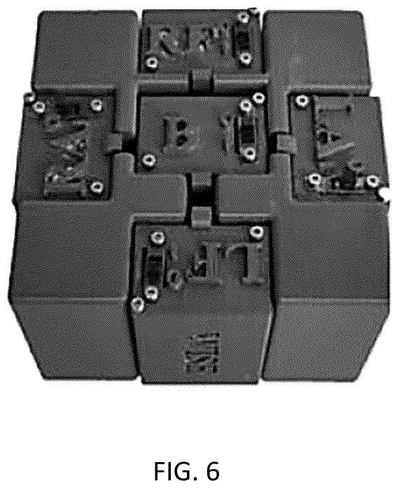

The WIS modules include tri-axial MARG sensors with each sensor yielding a calibration status ranging from zero (calibration not initialized) to three (all three axes calibrated). An intuitive design with horizontal progress bars is used to represent the calibration status for all the WIS modules. A calibration or sensor holder (FIG. 6) is designed to house the WIS modules and perform the calibration. In some embodiments, the calibration routine for each sensor comprises the following procedure: (i) the sensor holder is placed stationary for calibration of the gyroscope; (ii) the sensor holder is rotated to ≈45° about each axis for calibration of the accelerometer; and (iii) the sensor holder is randomly moved in 3D space for calibration of the magnetometer.

FIG. 7 shows an example of a sensor calibration user interface for visualization of sensor calibration status of the wearable inertial sensor modules each containing three MARG sensors. The users can utilize the visual feedback representing the calibration status of the sensors to perform the calibration routine swiftly. FIG. 7 shows the calibration UI with indicators for the WIS modules, each with three sensors, with horizontal progress bars that have a discrete resolution of zero (full grey), one/two (partial grey/green), and three (full green).

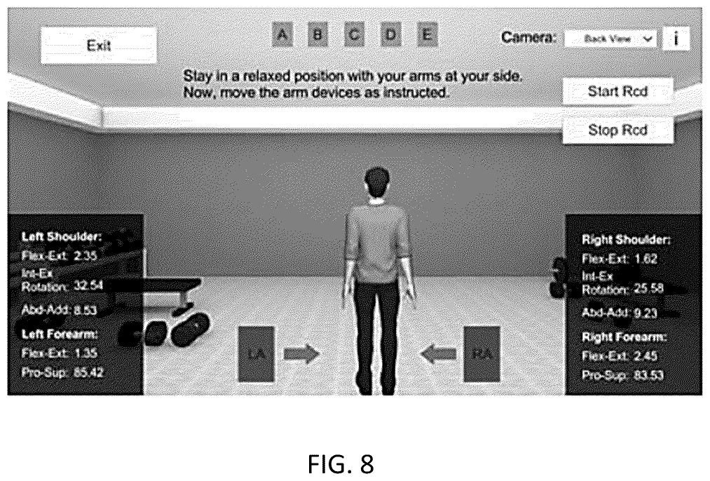

Precise sensor mounting on the human body is critical for accurate measurement of joint ROM. Prior literature has explored the use of standard initial position and determination of joint-to-sensor transformation using specific pre-determined movements prescribed to the subject. However, subjects with movement limitations may not be able to achieve a standard start pose or perform specific actions for anatomical calibration. An in-situ technique can be used to mount WIS modules to human body for accurate measurement of joint angles. The technique to mount the sensor on the forearm is intuitive due to the anatomical landmark created by the wrist joint on the forearm. However, the sensor placement on the upper arm segment proximal to the elbow requires precise mounting, which is difficult due to skin movements that result in erroneous internal-external rotation angles. As shown in FIG. 8, to address this challenge, a UI is created for the users to visualize all the JCS joint angles (shoulder: plane, elevation, and internal-external rotation; elbow: flexion-extension, pronation-supination, and carrying angle). Next, the orientation of the UE joints in 3D space is replicated by an animated human model and the UI provides visual cues for rotating the left arm (LA) and right arm (RA) sensors until shoulder internal-external rotation is within 5° for the neutral pose. These visual cues allow the user to adjust the LA and RA sensors to achieve precise alignment with the arm. FIG. 8 shows the sensor mounting UI with an animated model in neutral pose and the directional cues for the LA and RA sensors. In some embodiments, the interface shown in FIG. 8 is configured to facilitate the configuration and placement of sensors for each individual patient.

Accordingly, in some embodiments the present invention relates to methods of accurate sensor placement. The methods comprise steps of mounting and calibrating sensors in sequence, wherein a first sensor is used as a reference for accurate placement of a second sensor, the second sensor is used as a reference for accurate placement of a third sensor, and so on. In some embodiments, the methods comprise a first step of placing a first sensor on a subject. The first sensor can be placed at any location on an upper body of a subject, including but not limited to the lower back, upper back, nape of a neck, base of a neck, chest, sternum, stomach, navel, and the like. In some embodiments, the first sensor is placed in alignment with a subject's medial longitudinal axis. In some embodiments, the methods comprise a second step of placing a second sensor on each of a subject's left and right upper arms. In some embodiments, each of the second sensors is placed just proximal to a subject's left and right elbow joints. In some embodiments, the methods comprise a third step of placing a third sensor on each of a subject's left and right forearms. In some embodiments, each of the third sensors is placed just proximal to a subject's left and right wrist joints.

In some embodiments, the methods comprise a fourth step of adjusting a positioning of the second and third sensors based on real-time measurements of a subject. The real-time measurements include but are not limited to shoulder plane, shoulder elevation, shoulder internal-external rotation, elbow flexion-extension, elbow pronation-supination, and elbow carrying angle. The real-time measurements can be displayed on a user interface and anatomically depicted using an animated human model to facilitate adjustment. For example, positioning of the sensors is adjusted such that a carrying angle at the elbow joints is between about 8° and 20°, depending on a subject's gender. As would be understood by persons having ordinary skill in the art, a carrying angle is measured between a longitudinal axis of a humerus and a longitudinal axis of a forearm. In another example, positioning of the sensors is adjusted such that internal-external rotation at the shoulder joints is within about 5° of a neutral pose.

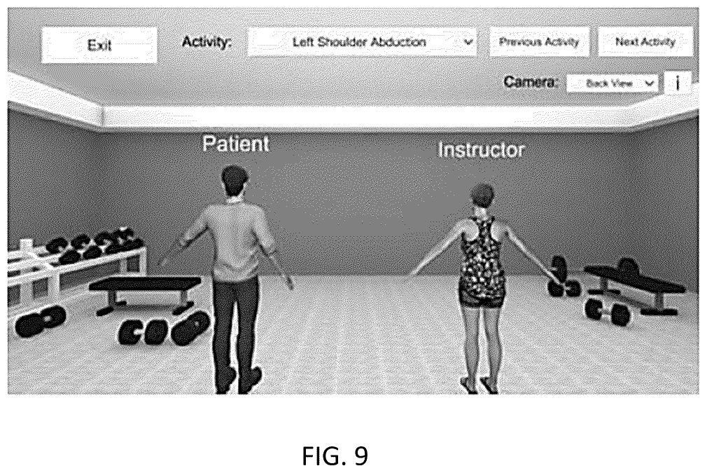

FIG. 9 shows a patient user interface with human models of patient and instructor performing shoulder abduction/adduction movements. A game environment emulating a virtual gym is developed for users to practice rehabilitation exercises. The virtual gym includes two human models that can be animated: (i) patient and (ii) instructor, both in the 3D environment allowing real time visualization of their movements. The WIS module's absolute quaternions are wirelessly streamed and converted to relative quaternions of the shoulder and forearm movements, which are in-turn utilized to compute the JCS-based joint angles and ROM. The UI facilitates a drop-down menu for selecting ROM exercises such as shoulder abduction/adduction, flexion/extension, forearm pronation/supination, etc. The relative quaternions are utilized to animate the patient model to provide real-time visual feedback on the movements being performed. The instructor model demonstrates the ROM exercise selected by the user. During a typical treatment session, the user observes the instructor model performing the selected ROM exercise and examines his/her own movements reflected on the patient model with the data streamed from the WIS modules. Since the movements are reflected on a standard model, the data is de-identified. The interface also provides different viewing angles such as front, back, left, and right views. A screenshot of the developed application showing the instructor and patient users from the back view is presented in FIG. 9. The data from the patient UI is captured in the JCS framework as quaternions and saved for off-line asynchronous playback and evaluation by the clinicians at their convenience.

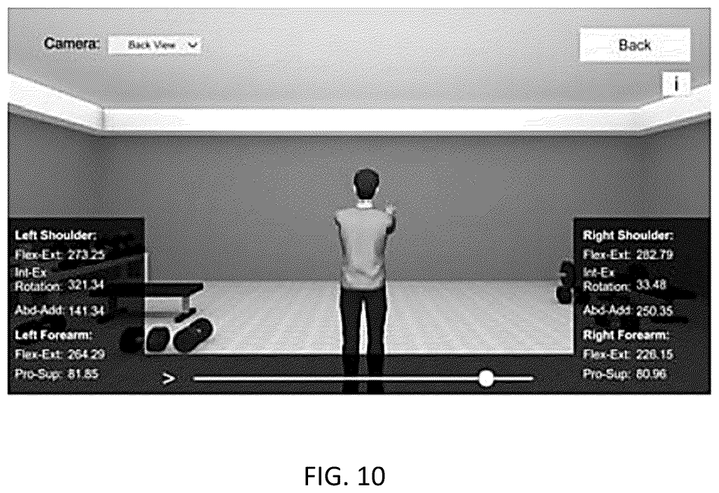

FIG. 10 shows a playback user interface for visualization of patient performance by the therapist. The playback UI facilitates the replay of the recorded ROM activity using the patient UI. The saved quaternion data is unpacked to create an interface similar to a media player with pause and play buttons. Additionally, a seek bar allows the clinician to navigate to specific temporal locations during the exercise for detailed examination of the movement. All the joint angles computed and saved during the exercise are displayed on the left and right panels corresponding to their respective joint angles. An information button “i” allows the user to toggle on/off the display of the joint angles. The camera view can be changed using the dropdown menu on top left (available views: back, front, left, and right). In FIG. 10, the playback interface shows a patient human model performing an exercise, the joint angles are displayed on each side.

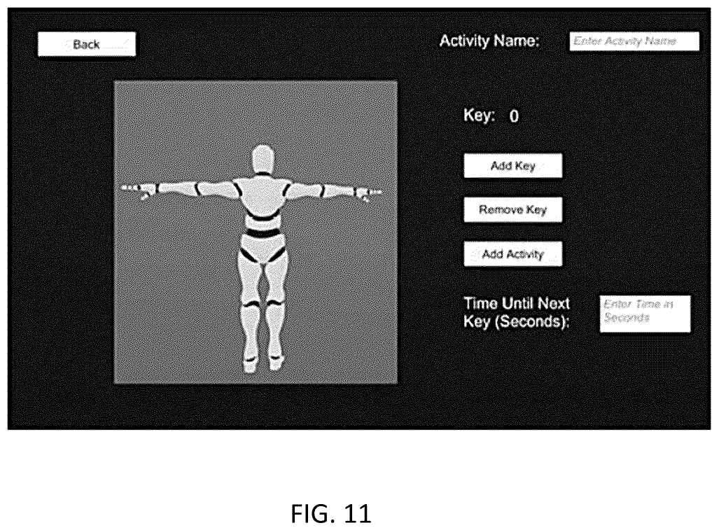

FIG. 11 shows an instructor user interface for creation of ROM exercises by therapists/clinicians. An instructor UI is designed for clinicians to develop exercises that are personalized for individual patients based on their therapy needs. This UI includes a virtual human skeletal model that utilizes the relative quaternions between the sensors to determine joint movements. It allows the user to enter the name of the exercise, select key points in the movement, and the time interval between the key points. Each key point saves joint positions of the UE enabling the user to create the desired exercises with very little effort. Once completed and saved, the exercise routine includes the arm passing through the key points, with a set time interval between key points. Spherical linear interpolation, a Unity3D built-in quaternion interpolation, is performed between the key points for the set time interval to facilitate a smooth movement between all the key points from start to end. A screenshot of the instructor UI with an exoskeleton human model for adding key points is shown in FIG. 11.

Each WISE module streams quaternion data of its orientation relative to the world coordinate frame ( [symbol] _{W}), which is represented by the direction of earth's gravity and the magnetic north pole. Quaternions are four-tuple objects that provide a computationally effective way to represent orientation. A quaternion q=(q_{w} qₓ q_{y} q_{z}) includes a scalar part q_{w} and a vector part [qₓ q_{y} q_{z}]^{T}. Three dimensional vectors are a subset of quaternions and a quaternion with its scalar part q_{w}=0 is termed a vector quaternion. Other forms of orientation representation include Euler angles, axis/angle representation, and rotation matrices. Consider the axis/angle representation (d,φ) where d=[dₓ d_{y} d_{z}]^{T} is the axis of rotation and φ is the angle of rotation, then the corresponding quaternion describing this rotation is given below.

$${q}_{d} ( φ ) = ( \begin{matrix}cos \frac{φ}{2} & {d}_{x} sin \frac{φ}{2} & {d}_{y} sin \frac{φ}{2} & {d}_{z} sin \frac{φ}{2}\end{matrix} ) \qquad ( 2 )$$

A vector V̅ expressed in a coordinate frame [symbol] ₁ (¹V̅) can be expressed in another coordinate frame [symbol] ₂ (²V̅) by using the quaternion product shown below.

q(²V̅)=¹q₂⊗q(¹V̅)⊗¹q*₂, q(V̅)=(0 V̅) (3)

where “⊗” and “*” denote the quaternion product and conjugate, q(V) denotes the vector quaternion of V̅, and ¹q₂ denotes the orientation of [symbol] ₂ relative to [symbol] ₁.

Although quaternions provide an efficient tool for orientation computation, they are unintuitive for interpretation by rehabilitation practitioners. In contrast, Euler angles represent rotations of one coordinate frame relative to another characterized by simple rotations about their principal axes. The joint coordinate system (JCS) framework, proposed by the International Society of Biomechanics, recommends the use of Euler angles for extracting anatomical joint angles (JA) for ease of use by practitioners. The UE JA measurements utilize the proximal coordinate frame as a reference to describe the angular rotation of the distal coordinate frame, i.e., the shoulder and elbow JA computations use the back and arm WISE modules, respectively, as references. To produce a reference for the shoulder JA computation, the back WISE module's quaternion is rotated and stored as q_{LBref} and q_{RBref} as below.

$${q}_{LBref} = {q}_{RBref} = {q}_{{Z}_{B}} ( - \frac{π}{2} ) ⊗ {q}_{B} \qquad ( 4 )$$

The sign convention for shoulder JA measurement is as follows: extension (−), flexion (+), adduction (−), abduction (+), external rotation (−), and internal rotation (+). To follow a similar sign convention, the axes of q_{LA}, q_{RA}, q_{LBref}, and q_{RBref} are flipped (see FIG. 1 where (·)† denotes the quaternions for the flipped coordinate frames). This axis flipping operation is performed by converting the quaternions to rotation matrices, followed by multiplication of −1 to the row vectors corresponding to the coordinate axes that are to be flipped. Throughout the axes flipping operation, care is taken to ensure that the rotations follow the right-hand rule. The relative quaternions between the shoulder and back's reference coordinate frame are computed as below.

q_{LS}q*_{LBref\}⊗q_{LA\} (5)

q_{RS}q*_{RBref\}⊗q_{RA\} (6)

To obtain the JA of the left and right shoulders, it is suggested the use of the Y−X−Y′ Euler angle convention. Since the WISE modules were assigned sensor coordinate frames consistently (FIG. 1), the appropriate placement and orientation of the LA and RA modules on arms required a slight adaptation of previous framework, leading to the use of Y−Z−Y′ Euler angle convention to obtain the JA of the left and right shoulders using the relative quaternions q_{LS} and q_{RS}. The quaternion to Euler angle conversion in the Y−Z−Y′ framework produces angles θ_{Y}, θ_{X}, and θ_{Y}+θ_{Y}, that represent the shoulder plane, shoulder elevation, and shoulder internal-external rotation angles, respectively. In the JCS framework, the shoulder elevation angle θ_{Z} relates to shoulder flexion-extension when θ_{Y}≈90° and to shoulder abduction-adduction when θ_{Y}≈0°.

The elbow JA computation utilizes the JCS framework to compute the ROM for flexion-extension and pronation-supination with the WISE system. Identical to the procedure of shoulder JCS ROM computation, the shoulder q_{LA} and q_{RA} are used to create references q_{LAref} and q_{RAref} as below.

$${q}_{LAref} = {q}_{{Y}_{LA}} ( \frac{π}{2} ) ⊗ {q}_{LA} \qquad ( 7 )$$

$${q}_{RAref} = {q}_{{Y}_{RA}} ( - \frac{π}{2} ) ⊗ {q}_{RA} \qquad ( 8 )$$

To conserve the standard sign convention of elbow joint movements flexion (+), pronation (+), extension (−), and supination (−), the coordinate axes of q_{LAref}, q_{RAref}, q_{LF}, and q_{RF} are flipped similar to the procedure for shoulders. The relative quaternions between the elbow and shoulder's reference coordinate frame are computed as below.

q_{LE}q*_{LAref\}⊗q_{LF\} (5)

q_{RE}q*_{RAref\}⊗q_{RF\} (6)

To obtain the elbow JA, it is suggested the use of the Z−X−Y Euler angle convention. Thus, using the relative quaternions q_{LE} and q_{RE} with the quaternion to Euler angle conversion in the Z−X−Y framework produces elbow joint angle θ_{Z}, θ_{X}, and θ_{Y} that correspond to the flexion-extension, carrying, and pronation-supination angles, respectively. The carrying angle is the angle between the humerus and the ulna, which is constant depending on the gender of the person ranging between 8° to 20°.

In some embodiments, the system facilitates the user to perform and visualize in real-time rehabilitation exercises. When used as a tele-rehabilitation system, it permits a therapist to review the key performance data from the rehabilitation session of user (playback interface) and suggest modified or additional exercise (instructor interface).

Further description of the system 100, UI and mathematics can be found in the following document included herein by reference in their entirety:

Rajkumar et al.,(2020), Usability study of wearable inertial sensors for exergames (WISE) for movement assessment and exercise. 1-15. 10.21037/mhealth-19-199.

### Rajkumar et al., Wearable Inertial Sensors for Range of Motion Assessment, IEEE SENSORS JOURNAL, VOL. 20, NO. 7, Apr. 1, 2020

### Bethi et al., Wearable Inertial Sensors for Exergames and Rehabilitation, 42nd Annual International Conference of the IEEE Engineering in Medicine & Biology Society (EMBC), Montreal, QC, Canada, 2020, pp. 4579-4582

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

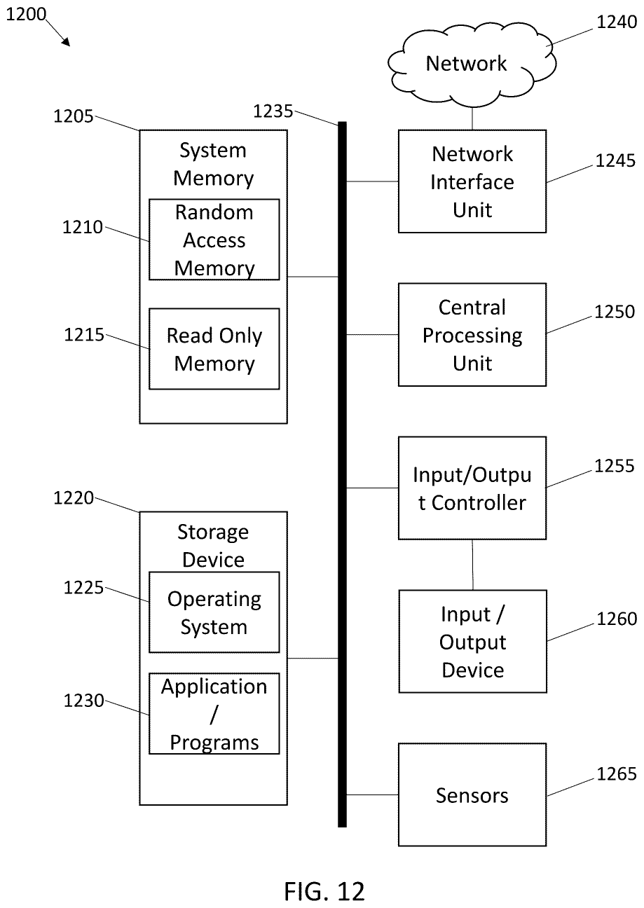

FIG. 12 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 12 depicts an illustrative computer architecture for a computer 1200 for practicing the various embodiments of the invention. The computer architecture shown in FIG. 12 illustrates a conventional personal computer, including a central processing unit 1250 (“CPU”), a system memoryl205, including a random access memory 1210 (“RAM”) and a read-only memory (“ROM”) 1215, and a system bus 1235 that couples the system memory 1205 to the CPU 1250. A basic input/output system containing the basic routines that help to transfer information between elements within the computer, such as during startup, is stored in the ROM 1215. The computer 1200 further includes a storage device 1220 for storing an operating system 1225, application/program 1230, and data.

The storage device 1220 is connected to the CPU 1250 through a storage controller (not shown) connected to the bus 1235. The storage device 1220 and its associated computer-readable media, provide non-volatile storage for the computer 1200. Although the description of computer-readable media contained herein refers to a storage device, such as a hard disk or CD-ROM drive, it should be appreciated by those skilled in the art that computer-readable media can be any available media that can be accessed by the computer 1200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1200 may operate in a networked environment using logical connections to remote computers through a network 1240, such as TCP/IP network such as the Internet or an intranet. The computer 1200 may connect to the network 1240 through a network interface unit 1245 connected to the bus 1235. It should be appreciated that the network interface unit 1245 may also be utilized to connect to other types of networks and remote computer systems.

The computer 1200 may also include an input/output controller 1255 for receiving and processing input from a number of input/output devices 1260, including a keyboard, a mouse, a touchscreen, a camera, a microphone, a controller, a joystick, or other type of input device. Similarly, the input/output controller 1255 may provide output to a display screen, a printer, a speaker, or other type of output device. The computer 1200 can connect to the input/output device 1260 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1220 and RAM 1210 of the computer 1200, including an operating system 1225 suitable for controlling the operation of a networked computer. The storage device 1220 and RAM 1210 may also store one or more applications/programs 1230. In particular, the storage device 1220 and RAM 1210 may store an application/program 1230 for providing a variety of functionalities to a user. For instance, the application/program 1230 may comprise many types of programs such as a word processing application, a spreadsheet application, a desktop publishing application, a database application, a gaming application, internet browsing application, electronic mail application, messaging application, and the like. According to an embodiment of the present invention, the application/program 1230 comprises a multiple functionality software application for providing word processing functionality, slide presentation functionality, spreadsheet functionality, database functionality and the like.

The computer 1200 in some embodiments can include a variety of sensors 1265 for monitoring the environment surrounding and the environment internal to the computer 1200. These sensors 1265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Additionally, data provided by the system 100 can be further analyzed and utilized by machine learning or artificial algorithms to detect specific patterns of joint movement/motion and, in certain instances, provide for tailored patient rehabilitation routines. Further, in one embodiment, data obtained from individuals using system 100 can be used to train the machine learning or artificial intelligence algorithms and model.

The invention further relates to a method of detecting upper extremity range of motion in terms of joint angles using system 100. In one embodiment, the method begins with calibrating the inertial sensors. In one embodiment, the method further includes correcting for gravity based and magnetic based misalignment of the sensors relative to Earth's gravitational and magnetics fields. Additionally, in one embodiment, the method includes collecting inertial sensor data. In one embodiment, the method further includes calculating relative quaternion positions between the inertial sensors and converting the calculated quaternion positions to joint angles. Additionally, in certain embodiments, the method includes displaying the joint angles to a user via a user interface.

The invention further relates to methods of diagnosis, prognosis, and/or monitoring treatment of upper body mobility diseases and disorders. The methods use the WISE system of the present invention to acquire ROM information from a subject to identify one or more joint locations having a deviation in ROM. In various embodiments, an affected joint includes but is not limited to the shoulder joint, elbow joint, and wrist joint. In some embodiments, the affected joint range of motion includes but is not limited to shoulder flexion, shoulder extension, shoulder abduction, shoulder adduction, shoulder internal rotation, shoulder external rotation, shoulder protraction, shoulder retraction, shoulder plane, shoulder elevation, elbow flexion, elbow extension, carrying angle, wrist pronation, wrist supination, wrist radial deviation, wrist ulnar deviation, wrist palmarflexion, wrist dorsiflexion, finger flexion, finger extension, finger abduction, finger adduction, thumb flexion, thumb extension, thumb opposition, thumb abduction, thumb adduction. The embodiment can be extended to include neck flexion, neck extension, neck rotation, next lateral bending, spine flexion, spine extension, spine lateral bending, spine rotation, hip flexion, hip extension, knee flexion, knee extension, ankle plantar flexion, ankle dorsiflexion, eversion, inversion, toe flexion, toe extension, and the like. In one embodiment, one or more ROM occurs in the sagittal plane, frontal plane, and/or horizontal planes.

In embodiment, the invention provides methods of diagnosing a subject as having a reduced range of motion, or a disease or disorder associated therewith, based on detection of one or more joints having decreased mobility. For example, certain diseases or disorders can be characterized by impaired mobility in a specific joint, impaired mobility within a specific range in a specific joint, impaired mobility in a specific combination of joints, impaired mobility within a specific range in a specific combination of joints, and the like. Contemplated diseases or disorders include but are not limited to stroke, multiple sclerosis, spinal cord injury, nerve damage, rheumatism, arthritis, fracture, sprain, stiffness, weakness, impaired coordination, impaired proprioception, pain, epicondylitis, tendonitis, and the like. In certain embodiments, the methods are useful in identifying one or more locations having an increased ROM, such that a subject can be characterized with a disease or disorder associated with joint hypermobility or compensatory movements that may be detrimental if not corrected.

Deviations in joint ROM are relative to a baseline joint ROM. In some embodiments, a baseline joint ROM comprises normative data, such as measurements from a global population, a regional group, an ethnic group, an age group, a gender group, and the like. In some embodiments, a baseline joint ROM comprises measurements from an individual subject. Baseline joint ROM can comprise measurements from healthy subjects, from subjects having a particular disorder or disease, from subjects at a stage of progression in a particular disorder or disease, or from subjects at a stage of treatment in a particular disorder or disease. In various embodiments, deviations can include but are not limited to increases or decreases in joint ROM by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and the like. The specific pattern and combination of increases and decreases may characterize specific conditions and provide the basis for diagnosis, prognosis, and therapeutic interventions.

In one embodiment, the invention provides methods of assessing progression and/or treatment of a disease or disorder in one or more joints of a subject by monitoring joint ROM over a period of time. The methods assess joint ROM before, during, and/or after the administration of treatment. In some embodiments, the methods of the invention further include steps of administering a treatment regimen to a subject identified as having a reduced range of motion. In some embodiments, a subject is administered a composition for treatment of a disease or disorder associated with a reduced range of motion. In some embodiments, a subject is administered exergaming intervention using the exergaming embodiment for increased or decreased range of motion at one or more joints. In some embodiment a subject is administered physical therapy for treatment of a disease or disorder associated with a reduced or increased range of motion at one or more joints. In some embodiments, a subject is administered injection intervention for treatment of a disease or disorder associated with a reduced or increased range of motion at one or more joints. In some embodiments, a subject is administered surgical intervention for treatment of a disease or disorder associated with a reduced or increased range of motion at one or more joints. In some embodiments, a subject is administered an orthopedic device for treatment of a disease or disorder associated with a reduced or increased range of motion at one or more joints.

In various embodiments, the methods of diagnosis, prognosis, and/or monitoring treatment of upper body mobility diseases and disorders are implemented with a user interface comprising a virtual subject representing an actual subject, a virtual coach demonstrating movements intended for a subject to mimic, real-time displays of joint position and ROM, and combinations thereof. The user interface can be used locally or remotely, such as in telemedicine. In some embodiments, the user interface comprises programmable instructions wherein a doctor or instructor provides a series of movements for a subject to follow or mimic, wherein the series of movements induces the subject to move one or more joints within a specified ROM such that one or more diseases or disorders are revealed if the subject is unable to achieve a baseline joint ROM or is unable to achieve a joint ROM within a range of a baseline joint ROM.

The invention further relates to a computer-readable medium utilized to facilitate detection of upper extremity range of motion. In one embodiment, the computer-readable medium provides an exergame utilizing live joint angle calculations. The computer-readable medium includes a computer program code segment used to communicate with a plurality of inertial sensors, a computer program code segment used to calibrate the plurality of inertial sensors, a computer program code segment used to collect inertial sensor data, a computer program code segment used to calculate relative quaternion positions between the inertial sensors, a computer program code segment used to convert the calculated quaternion positions to joint angles, and a computer program code segment used to present the joint angles on a exergame user interface.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Calibration Results

To validate the effectiveness of the developed Uls, two experiments were conducted which document the improvements in (i) calibration time and (ii) time taken for sensor mounting on the human body as well as (iii) examination of subject adherence to instructor-programmed exercise routines.

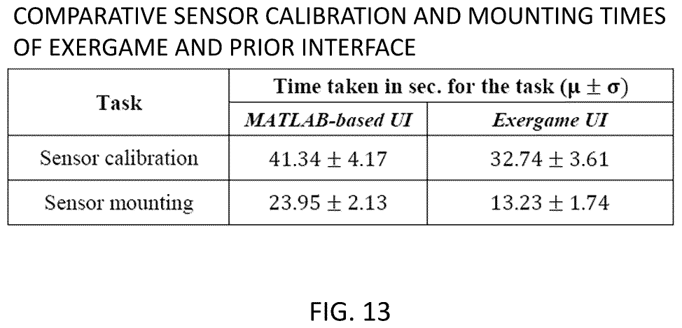

Text output is utilized to observe the calibration status. To quantify the effectiveness of the newly developed calibration UI, the time taken for calibration between the prior approach, wherein one observes calibration status from text output communicated via serial port, versus the use of the calibration UI was compared. With the UI, calibration time was initialized to zero upon turning-on the devices and the completion time was determined upon successful calibration of all the five WIS modules. The procedure was repeated for five trials and the resulting mean μ and standard deviation σ for the time taken with both approaches are given in FIG. 13.

A MATLAB-based real time animated plotting interface was created to visualize the joint angles, and based on the resulting plot sensor mounting was adjusted. To evaluate the effectiveness of visual cues for sensor mounting on the human body, the time taken for sensor mounting between the MATLAB-based interface versus the newly created sensor mounting U was compared. The time taken was recorded from the start of the UI to the successful alignment of the device, i.e., once internal-external rotation reaches within ±5°. The procedure was repeated for five trials and the resulting μ and σ of the time recorded with both approaches are provided in FIG. 13.

The results in FIG. 13 indicate that the exergame interfaces improve the system performance. An experimental trial was conducted to assess the subject's adherence to an instructor programmed exercise routine involving shoulder abduction/adduction with maximum ROM of ˜90° for six trials. Using the patient UI, the subject performed six repetitions of the exercise. The ROM angle achieved by the subject across six repetitions was found to have μ+σ of (97.34±5.12)°.

### Earth's Gravity-Based Misalignment Correction

The BNO055 sensor measures absolute orientation of the sensor relative to the world coordinate frame ( [symbol] _{W}) whose \hat{Z}_{W}-axis is anti-parallel to gravity and Ŷ_{W}-axis points towards the magnetic north of earth. Preliminary measurements revealed that after soldering BNO055 to the PCB and placing it inside the housing, the BNO055 sensor's coordinate frame ( [symbol] _{S}) was not aligned with the coordinate frame of the 3D-printed housing ( [symbol] _{H}). To correct the orientation misalignment between the two frames, the [symbol] _{W} was utilized as a reference to develop two software signal processing methods: (i) earth's gravity (EG approach) and (ii) earth's magnetic field and gravity (EGM approach) to transform [symbol] _{W} and align it with [symbol] _{H}.

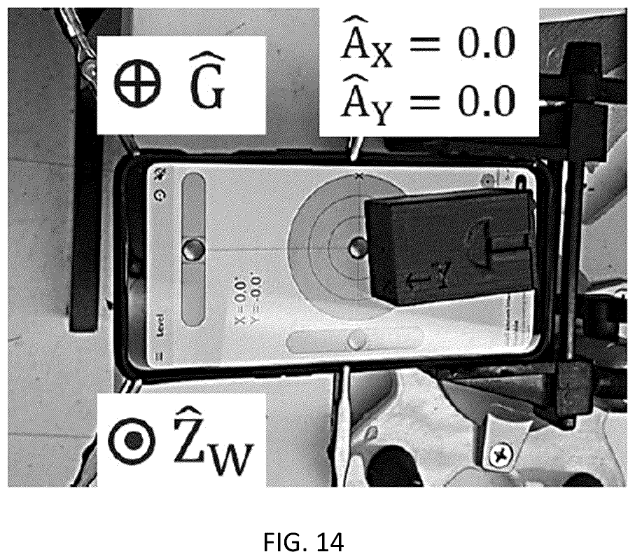

The sensor fusion algorithm embedded in the BNO055 MARG sensor is based on the principle that whenever a rotational axis of the sensor is aligned with anti-parallel to the earth's gravity vector (\hat{Z}_{W}) the angular rotation measurements about the other two axes are 0°. The direction of the vector \hat{Z}_{W}, anti-parallel to gravity, was utilized as a reference for correcting the BNO055 orientation [symbol] _{S} and obtain [symbol] _{H}. A flat table was created by using a smartphone's accelerometer such that its screen is normal to gravity. The 3D-printed housing was placed on this table (see FIG. 4) with its \hat{Z}_{H}-axis (\hat{Z}_{H}) pointing upward and thus parallel to \hat{Z}_{W}. As seen in FIG. 14, the accelerometer measurements for the X- and Y-axes of the smartphone are zero indicating that the gravity vector is normal pointing inward to the smartphone's screen and \hat{Z}_{W} is pointing outward.

The Z s -axis of the sensor, expressed in the sensor frame, is given by \tilde{Z}_{S}=[0 0 1]^{T} and \tilde{q}(\tilde{Z}_{S})=(0\tilde{Z}_{S}^{T}) denotes the quaternion corresponding to \tilde{Z}_{S}. Now, the \tilde{Z}_{S} vector is expressed in [symbol] _{W} as a quaternion as shown below.

\hat{q}_{Z}_{_{S}}=\hat{q}_{S}⊗\tilde{q}(\tilde{Z}_{S})⊗\hat{q}*_{S} (11)

Using the above formula, the Z_{S}-axis of the sensor in [symbol] _{W} can be extracted as \hat{Z}_{S}=\hat{V}(\hat{q}_{Z}). In the above formula, \hat{q}_{S}denotes the quaternion measurement obtained from the BNO055 sensor, i.e., its orientation relative to [symbol] _{W}. To obtain \hat{Z}_{S}, ideally, \hat{Z}_{S} is expected to align with the direction of \hat{Z}_{W}(or \hat{Z}_{H}), which is given by \hat{Z}_{W}=[0 0 1]^{T}. However, human errors cause unavoidable misalignment in the orientations of the sensor (\hat{Z}_{S}) and the housing (\hat{Z}_{H}). This results in a non-zero angle γ between \hat{Z}_{S} and \hat{Z}_{W}, which can be computed by using the dot product below.

γ=cos⁻¹(\hat{Z}_{S}·\hat{Z}_{W}) (12)

A software rotation of γ needs to be performed to align \hat{Z}_{S} to \hat{Z}_{H} and this rotation needs to be performed only around a vector normal to \hat{Z}_{S} and \hat{Z}_{W}. Thus, a vector \hat{P} was determined, normal to \hat{Z}_{S} and \hat{Z}_{W}, and computed in [symbol] _{W} as shown below.

\hat{P}=\hat{Z}_{W}×\hat{Z}_{S} (13)

Using \hat{P}, \tilde{P} was computed by expressing it in [symbol] _{S} and it is found to be constant throughout the entire 360° rotation of the housing about \hat{Z}_{H}. The \tilde{P} obtained is retained for further processing. The alignment of Z_{S}-axis to the Z_{H}-axis requires the rotation of the [symbol] _{S} by γ about the \hat{P} axis. Hence, the pure quaternion of \tilde{P} is expressed in [symbol] _{W} as \hat{q}(\hat{P}_{S}) as shown below.

\hat{q}(\hat{P}_{S} )=\hat{q}_{S}⊗\tilde{q}(\hat{P}_{S} )⊗\hat{q}*_{S} (14)

Next, the quaternion \hat{q}_{\hat{P}}(γ) describing a rotation in [symbol] _{W} of angle γ around \hat{P}_{S}=[x y z]^{T} is computed. Finally, for the updated coordinate frame of sensor ( [symbol] _{S′}), \hat{q}_{S′} whose \hat{Z}_{S′}, is aligned with \hat{Z}_{H} was computed.

\hat{q}_{S′}=\hat{q}*_{\hat{P}}(γ)⊗\hat{q}_{S} (15)

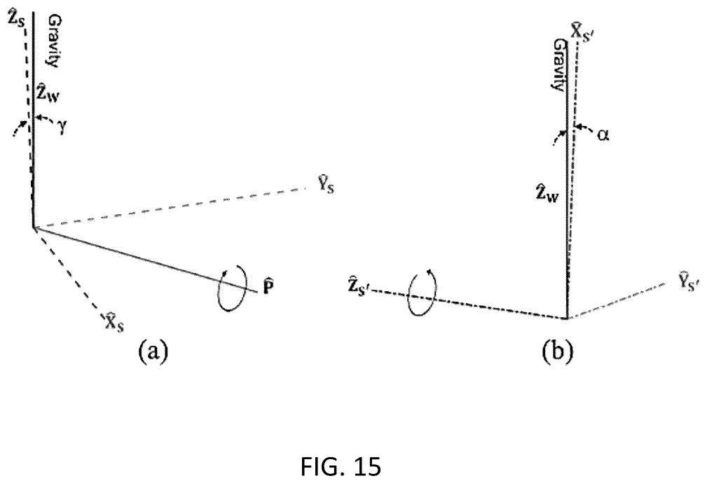

This rotation is pictorially represented in FIG. 15A.

Next, the housing is placed with its X_{H}-axis (\hat{Z}_{H}) aligned with \hat{Z}_{W} and the corresponding angle a between the \hat{X}_{H}, and \hat{Z}_{W} is determined. The \hat{Z}_{S′}-axis of the sensor in [symbol] _{W}, i.e., V(q_{Z′})=[x′ y′ z′]^{T}, is computed using \hat{q}_{S′}. The quaternion for rotating the sensor about \hat{Z}_{S′} axis through an angle α is given by \hat{q}_{\hat{Z}′}_{_{S}} (α) and computed. The new quaternion \hat{q}_{H} represents the absolute orientation of the housing and is obtained from the formula below.

\hat{q}_{H}=\hat{q}*_{\hat{Z}′}_{_{S}}(α)⊗\hat{q}_{S′} (16)

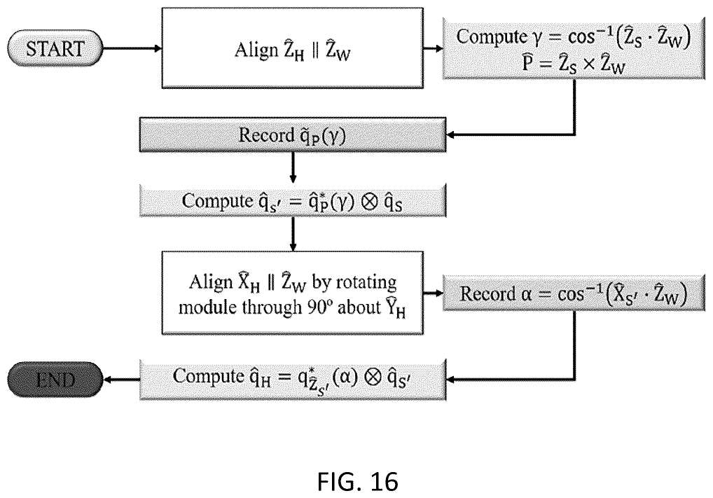

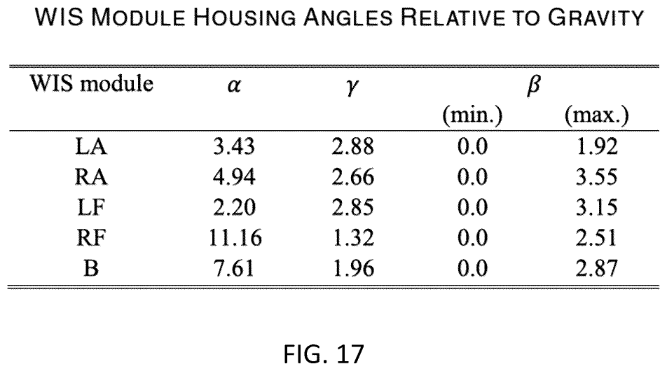

Finally, the EG approach is validated by placing the sensor on the phone's screen with Y_{H}-axis pointing upward and angle β between Ŷ_{H}-axis and the \hat{Z}_{W}-axis is computed for verification. Table 1 lists the α, β, and γ angles obtained from the five WIS modules. A flow chart of the steps involved in EG-based orientation misalignment correction is shown in FIG. 16. FIG. 17 shows the α, β, and γ angles obtained from the five WIS modules.

### Earth's Gravity and Magnetic Field-Based Correction

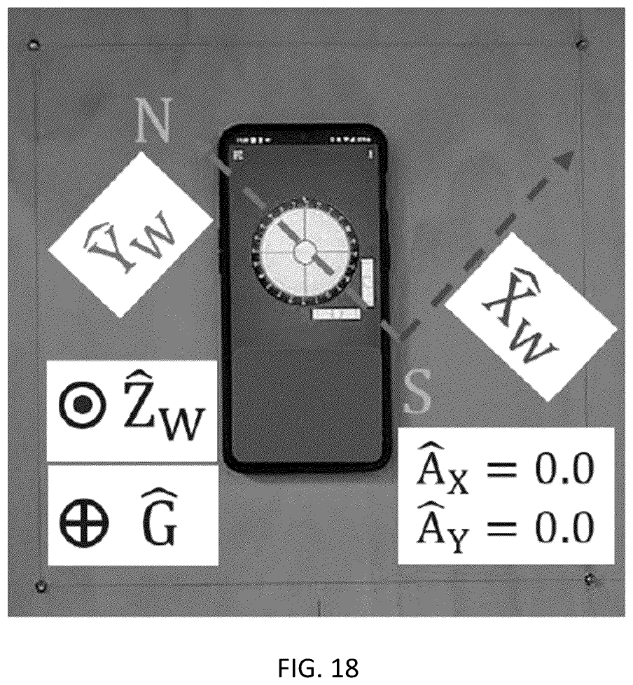

A smartphone screen as a flat surface is not suitable for orientation correction with the EGM approach, since smartphones utilize electromagnetic waves for communication that disturb sensor measurements. Hence, a wooden platform was created with adjustable screws to perform the correction. The calibrated wooden platform is shown in FIG. 18. Here the gravity vector pointing inward is normal to the wooden platform and \hat{Z}_{W} points outward. A marking is made on the wooden platform based on the magnetic north pole direction, representing Ŷ_{W}, shown by the smartphone's compass.

From the principle of BNO055′s sensor fusion algorithm, the measurement of absolute orientation of the sensor is relative to [symbol] _{W}. Specifically, when the sensor is oriented such that the direction of Z s -axis is along 2 w and the Y s -axis is aligned with Ŷ_{W}, the sensor provides zero measurements in yaw, pitch, and roll. This information is utilized to align the [symbol] _{H} such that its Y H -axis is parallel to the earth's magnetic field Ŷ_{W} and Z_{H}-axis parallel to \hat{Z}_{W} (i.e., [symbol] _{H} and [symbol] _{W} are now aligned). Ideally, [symbol] _{S} is expected also to align with Yw. However, due to misalignment between [symbol] _{H} and [symbol] _{S}, \hat{q}_{S}≠[1 0 0 0].

Now, a measurement of quaternion for sensor orientation is obtained (in Yw) and its conjugate is saved as \tilde{q}*_{S}. It can be shown that \tilde{q}_{H}=\tilde{q}*_{S}, where \tilde{q}_{H} denotes the quaternion of the housing represented in [symbol] _{S}. Next, the housing orientation \tilde{q}_{H} is expressed in [symbol] _{W} using \tilde{q}_{H}=\hat{q}_{S}⊗\tilde{q}_{H}⊗\tilde{q}_{S}. Now, the sensor misalignment is corrected by applying the rotation \tilde{q}_{H} to the sensor measurement \hat{q}_{S} using \hat{q}_{S′}, =\hat{q}_{H}⊗\hat{q}_{S}. A 90° rotation about the Y_{H}-axis, aligns the X_{H}- axis with \hat{Z}_{W}. However, if [symbol] _{H} is not coincident with [symbol] _{S}, the resulting X_{S′}-axis and \hat{Z}_{W}will have a non-zero static offset angle θ, which can be computed as below.

θ=cos⁻¹(\hat{X}_{S′}·\hat{Z}_{W}) (17)

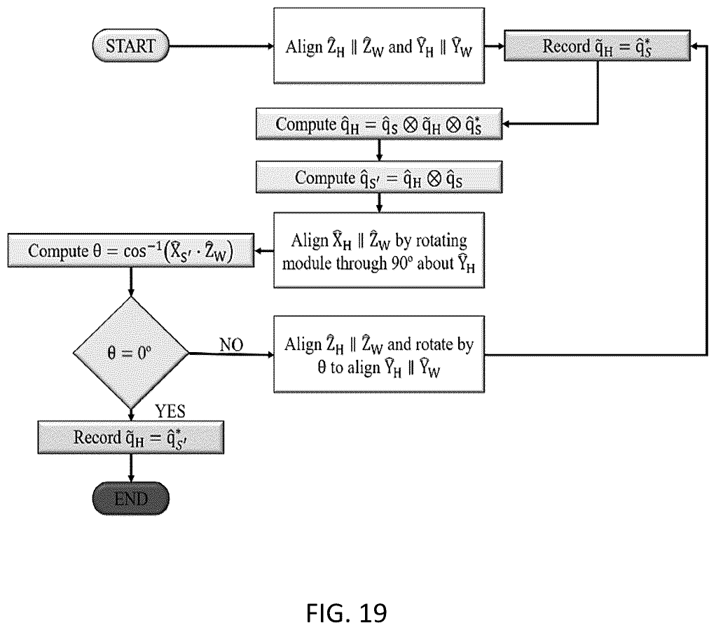

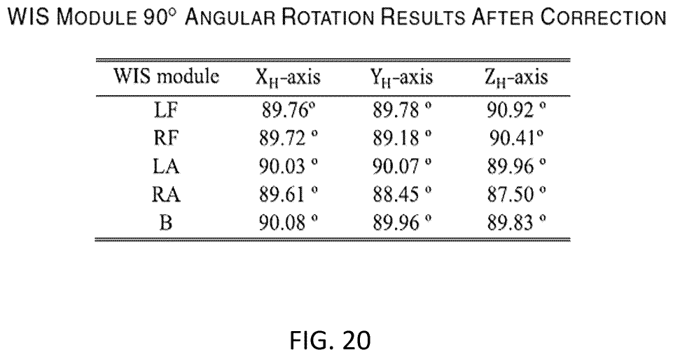

The angle θ is the misalignment due to the error in aligning the Y_{H}-axis to Ŷ_{W}. The housing was reverted to its old position where Z_{H}-axis is parallel to \hat{Z}_{W} and rotate the housing by an angle θ to align the Y_{H}-axis with Ŷ_{W}. The sensor datar at this stage provides the alignment of the housing with [symbol] _{W}. Thus, as per above, measurement of quaternions for sensor orientation is obtained and its conjugate \tilde{q}*_{S′}, is saved as \tilde{q}_{H}. The steps for the correction procedure are delineated in the block diagram shown in FIG. 19. The effectiveness of the algorithm is validated by performing a rotation of 90° about the housing's principal axes. The measured rotation angle for the principal axes of each WIS module's housing are reported in FIG. 20.

### Joint Coordinate System (JCS)

The quaternions are an effective representation for rotation and computation in 3D space, however, they are rarely used to characterize ROM measurements by therapists and clinicians. The JCS is a standard reporting method proposed by the ISB for computing human joint angles. Furthermore, reporting results using a single standard allows transparent communication between researchers and clinicians. The JCS method uses the proximal coordinate frame as a reference to define the joint angle of the distal coordinate frame. The shoulder joint angles use the thorax coordinate frame as the reference and the elbow joint angles use the shoulder coordinate frame as the reference. The coordinate frames and corresponding relative joint angles are described from a starting neutral pose (NP) as shown in FIG. 1.

In the JCS implementation of the WIS system presented herein, the back-sensor module B is used as a reference for LA and RA sensor modules to compute the shoulder joint angles. Similarly, the LA and RA sensor modules are used as references for the LF and RF sensor modules, respectively, to compute the elbow and forearm movements. For the shoulder angle computation, an initial reference is needed for the back inertial sensor module at NP. To do so, two quaternions q_{RBref} and q_{LBref} are created as shown below

$${q}_{RBref} = {q}_{LBref} = {\hat{q}}_{{\hat{Z}}_{B}} ( - \frac{π}{2} ) ⊗ {\hat{q}}_{B} \qquad ( 18 )$$

The sign convention of shoulder joint angle measurements is defined as extension (−) and flexion (+), adduction (−) and abduction (+), and external (−) and internal (+) rotation.

The axes shown in FIG. 1 for the \hat{q}_{LA}, \hat{q}_{RA}, q_{RBref}, and q_{LBref} are rotated by 180° to achieve a similar sign convention. All the rotated coordinate frames are pictorially represented in FIG. 1. The quaternion representing the rotation of the shoulder relative to the back WIS module is extracted for the left and right sides, where (·)† denotes the quaternions for the rotated coordinate frames.

q_{LS}=q*_{LBref\}⊗q_{LA\} (19)

q_{RA}=q*_{RBref\}⊗q_{RA\} (20)

The Y−X−Y′ Euler angle convention is was previously used to obtain the shoulder joint angles. Since the orientation of the LA and RA WIS modules differ from previous use, Y−Z−Y′ Euler angle convention is adopted. The joint angles are computed using MATLAB's built-in command quat2angle from q_{LS} and q_{RS}. The quat2angle command returns angles θ_{Y}, θ_{Z}, and θ_{Y}+θ_{Y′} that represent rotation in the shoulder plane, shoulder elevation, and shoulder internal-external rotation, respectively. Shoulder elevation θ_{Z} refers to shoulder flexion-extension (in the sagittal plane) when θ_{Y}≈90° and to shoulder abduction-adduction (i.e., the frontal plane) when θ_{Y}≈0°.

The JCS implementation for measuring elbow rotation requires the use of left arm (LA) and right arm (RA) inertial sensors as references, i.e., q_{LAref} and q_{RAref}, respectively, which are computed as below.

$${q}_{LAref} = {\hat{q}}_{{\hat{Y}}_{LA}} ( \frac{π}{2} ) ⊗ {\hat{q}}_{LA} \qquad ( 21 )$$

$${q}_{RAref} = {\hat{q}}_{{\hat{Y}}_{RA}} ( - \frac{π}{2} ) ⊗ {\hat{q}}_{RA} \qquad ( 22 )$$

The sign convention for the elbow and forearm measurements are defined as extension (−) and flexion (+) and supination (−) and pronation (+). As per above, the axes of the coordinate frames q_{RAref}, q_{LAewf}, q_{LF}, and q_{RF} are rotated by 180° to achieve a similar sign convention. The relative quaternions representing the left (q_{LE}) and right (q_{RE}) elbow joint angles are computed as below.

q_{LE}=q*_{LAref\}⊗q_{LF\} (23)

q_{RE}=q*_{RAref\}⊗q_{RF\} (24)

Next, the Z-X-Y Euler angle convention is used to obtain the left and right elbow joint angles by using quat2angle MATLAB command from q_{LE} and q_{RE}, respectively. The quat2angle command returns angles θ_{Z}, θ_{X}, and θ_{Y} that indicate elbow flexion-extension, carrying, and pronation-supination angles, respectively. The carrying angle is the angle between the humerus in the upper arm and the ulna in the forearm, which ranges between 8° to 20°.

### WIS Mounting and Alignment

Mounting the sensors at the distal end of the limb segment reduces most errors in measurement. For example, the forearm sensors (LF and RF) are placed proximal to the wrist joint to produce acceptable results for elbow rotation. However, even when the arm sensors (LA and RA) are placed just proximal to the elbow joint, they are prone to erroneous measurements of internal-external rotation at the shoulder due to skin movements. Thus, correct mounting of the WIS modules is critical for accurate measurement of joint ROM. Inertial sensors have previously been calibrated by using a standard initial position and a prescribed motion to correct for mounting uncertainties. However, patients with motor deficits may not be able to achieve these initial positions or perform prescribed movements to produce the suggested joint-to-sensor transformation. Hence, as an alternative, an in-situ solution was developed for accurate placement of sensors that is applicable to patients with real-world movement constraints. Specifically, the sensors LA, RA, LF, and RF are placed at their corresponding distal joint segments as shown in FIG. 1. The carrying angle at the elbow joints and the internal-external rotation at the shoulder joints are displayed in real-time during mounting of the sensors. The sensors are placed correctly when the carrying angle is reflected accurately based on the subject's gender (8°-20°) and internal-external rotations of the LA and RA sensors read zero. This directed real-time mounting strategy can permit correct positioning of sensors without the need to achieve any specific initial position or perform prescribed movements and does not require training in MOCAP.

### Experimental Validation

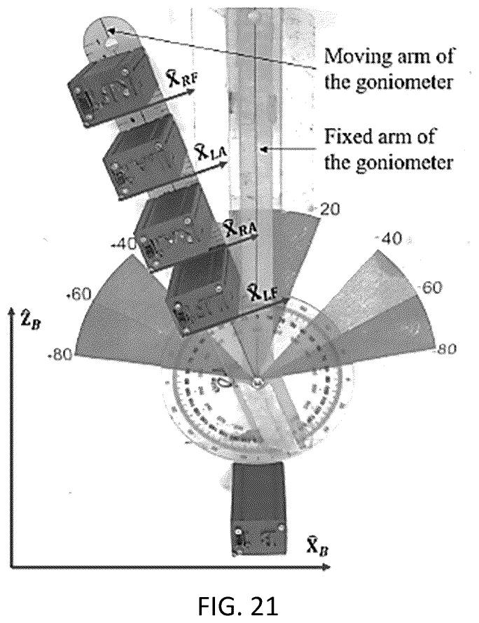

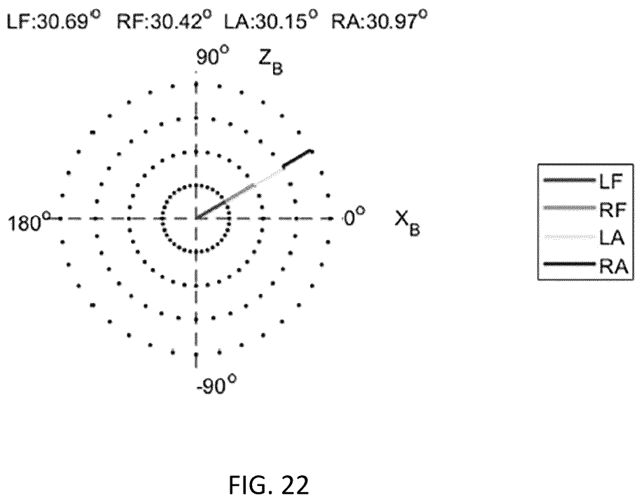

As evidenced above, the JCS approach utilizes the relative measurements between two WIS modules for computing the joint angles of the shoulder and elbow. Before conducting experimental measurements with a human subject using the WIS system, the accuracy of the relative angles was validated between the WIS modules by creating an experimental setup. Specifically, a 12-inch 360° clinical goniometer (Elite Medical Instruments, Orange County, CA) was mounted on a flat table to create a rotating platform (i.e., turntable) for testing the measurement accuracy of WIS modules. Next, four WIS modules (LA, RA, LF, and RF) were mounted on the moving arm of the goniometer and the WIS module B was fixed on the table parallel to the 0° start position of the other four WIS modules as shown in FIG. 21. A MATLAB user interface was created for data acquisition and visualization of the WIS modules' relative angles as shown in FIG. 22. To validate the angular measurement stability of BNO055, the temporal variability of sensor measurements was examined with an arbitrary fixed pose and with dynamic changes to it. Specifically, the relative orientations of the LF, RF, LA, and RA sensors vs. B sensor were measured for 300 sec. for both fixed (0°) and changing orientations (−90°, 0°, 90°, 180°). The resulting measurements exhibited a stable response with no drift or deviations.

Next, for each angular measurement, the movable arm of the goniometer was rotated manually from the 0° start position to a pre-determined target angle for ten trials. To test the measurement accuracy of a WIS module about each of its three axes of rotation, the module was placed on the turntable with the axis under test being orthonormal to the turntable. In this manner, the sensor data from each axis of the WIS modules was measured for various angular positions applied on the goniometer.

### Angular Accuracy Testing

The moving arm of the goniometer was manually rotated from 0° start position, in intervals of 20°, to various angular positions ranging between ±80°. The angular orientations of WIS sensor modules (LA, RA, LF, and RF) relative to WIS sensor module B were computed using two methods: (i) vector projection method for the EG approach and (ii) Euler angle method for the EGM approach. These two angular computation methods are applicable to measurements obtained from both the EG and EGM approaches and are included here only for illustration.

In the vector projection method, the EG approach was utilized to obtain the orientation of the housing from the sensor measurements. Each axis [symbol] , where [symbol] ∈{LA, RA, LF, RF} and Ω∈{X, Y, Z }, of the sensor module [symbol] was aligned with \hat{X}_{B}. Now, for each SZ E {X, Y, Z }, the rotation of sensor module [symbol] , about the axis normal to the turntable, was computed by projecting [symbol] on the X_{B}-Z_{B}plane of the WIS B module. For example, in FIG. 21, [symbol] ∥\hat{X}_{B} in the start position and the angular rotation of sensor module [symbol] was computed about [symbol] axis by projecting [symbol] on the X_{B}-Z_{B}plane. The angular rotation [symbol] of the WIS module [symbol] relative to the WIS module B was computed by using the atan2 function as shown below

[symbol]

=atan2(V)( [symbol] )·V(\hat{q}_{\hat{Z}}_{_{B}}), V( [symbol] )·V(\hat{q}_{\hat{X}}_{_{B}})) (25)

The vectors required to compute [symbol] were obtained from \hat{q}_{(·)}. The procedure included 10 trials for each angle between ±80° at 20° intervals was repeated for each axis of the WIS module.

In the Euler angle method, the EGM approach was utilized to obtain the orientation of the housing from the sensor measurements. A similar procedure as outlined above was repeated; however, the relative angles were computed using the relative quaternion [symbol] between the WIS module B and WIS modules [symbol] attached to the moving arm of the turntable as below.

q_{Rel} [symbol] =\hat{q}*_{B}⊗\hat{q} [symbol] (26)

Furthermore, the quat 2 angle MATLAB command was used to extract the relative angle R_{angle} for the axis tested using Tait Bryan's angle sequence, wherein the axis tested is the last axis of the sequence, i.e. Z axis testing can utilize X-Y-Z or Y-X-Z sequences. The procedure included 10 trials for each angle between ±80° at 20° intervals was repeated for each axis of the WIS modules.

### Experimental Results

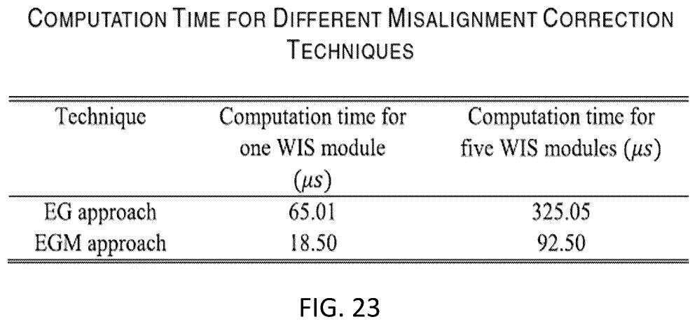

The computational time taken for the EG and EGM methods used for orientation misalignment correction techniques were computed using the MATLAB commands tic and toc and the results are presented in FIG. 23. The results indicate that the EGM approach, requiring three quaternion products, is computationally efficient compared to the EG approach, requiring six quaternion products.

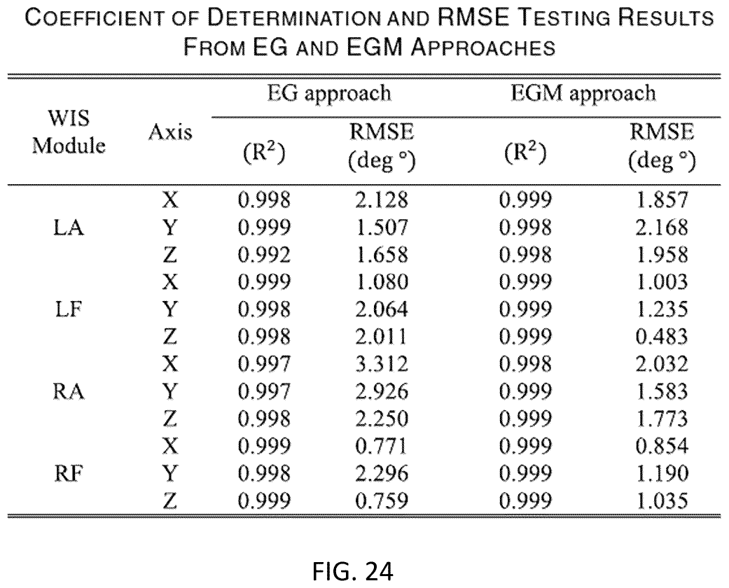

A MATLAB routine was developed to obtain the positive and negative peaks of the time series WIS module data using f indpeaks command. The peaks represent the measured angle ψ_{M} and were compared with the applied angle ψ_{A} on the goniometer. Coefficient of determination (R²) and the root mean square error (RMSE) between ψ_{A} and ψ_{M} of each WIS module using the two methods are presented in FIG. 24.

The data indicate an excellent correlation between the measured and applied angles. Furthermore, the high correlations indicate that the housing's coordinate frame computed from the sensor's coordinate frame were sufficiently accurate to measure ROM.

The accuracy and repeatability of sensor measurements are key parameters to describe the operating constraints of any measurement system. The accuracy [symbol] of the WIS module [symbol] expressed as average percentage deviation from the applied angle is given by

$${a}_{ℓ} := \pm \frac{1}{n} {\sum }_{i = 1}^{n} \frac{\vert {ψ}_{M {ℓ}_{i}} - {ψ}_{A {ℓ}_{i}} \vert}{\vert {ψ}_{A {ℓ}_{i}} \vert} \times 100 , \\ where n = {10}_{trials} \times {9}_{angles} \times {3}_{axes} .$$

The repeatability of the WIS module [symbol] is expressed as the coefficient of variation

$${C}_{{V}_{ℓ}} := {max}_{j = 1}^{m} ( \frac{\vert {σ}_{{ℓ}_{j}} \vert \times 100}{\vert {μ}_{{ℓ}_{j}} \vert} ) , where m = {9}_{angles} \times {3}_{axes}$$

and [symbol] and [symbol] are the mean and standard deviation, respectively, of ψ_{M} [symbol] for ten trials.

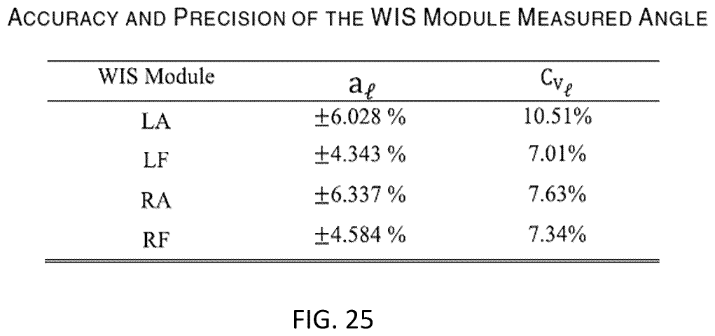

The computed values [symbol] and [symbol] for each WIS module relative angle are presented in FIG. 25. The results indicate that the relative angles obtained from the sensor modules are accurate within ±6.5% of measured angle and the small values of [symbol] indicate that the sensors produce repeatable results.

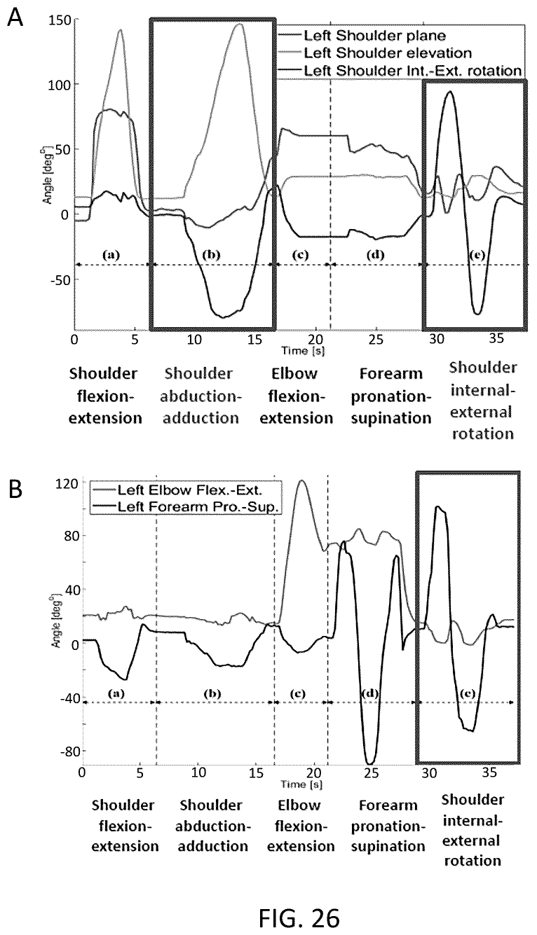

Having used the goniometer-based turntable described above for validating the relative measurements produced by the WIS modules, next the WIS modules were utilized for the JCS-based ROM measurements. Specifically, the WIS modules were mounted on a healthy human subject. The subject was asked to perform simple ROM exercises in the following order: (i) shoulder flexion/extension, (ii) shoulder abduction-adduction, (iii) elbow flexion-extension, (iv) forearm pronation-supination, and (v) shoulder internal-external rotation. The JCS method was used to compute joint angle measurements from the sensor data obtained from the shoulder (LA, RA), elbow (LF, RF), and back sensor (B). The JCS-based tri-planar motion for the shoulder is shown in FIG. 26A, where movement in the shoulder plane (frontal plane) is ≈90° when the shoulder is flexing and extending in the sagittal plane, and it is ≈0° when the shoulder is abducting and adducting in the frontal plane. Furthermore, during abduction-adduction, it is anatomically infeasible to move the shoulder beyond 90° without external rotation (which occurs in the horizontal plane). Similarly, note the elbow and forearm movements in FIG. 26B. These data illustrate that the integration of the JCS technique with the WIS sensor modules provides comprehensive information about joint motion in all three planes simultaneously. This is quite informative to understand movement limitations in patients.

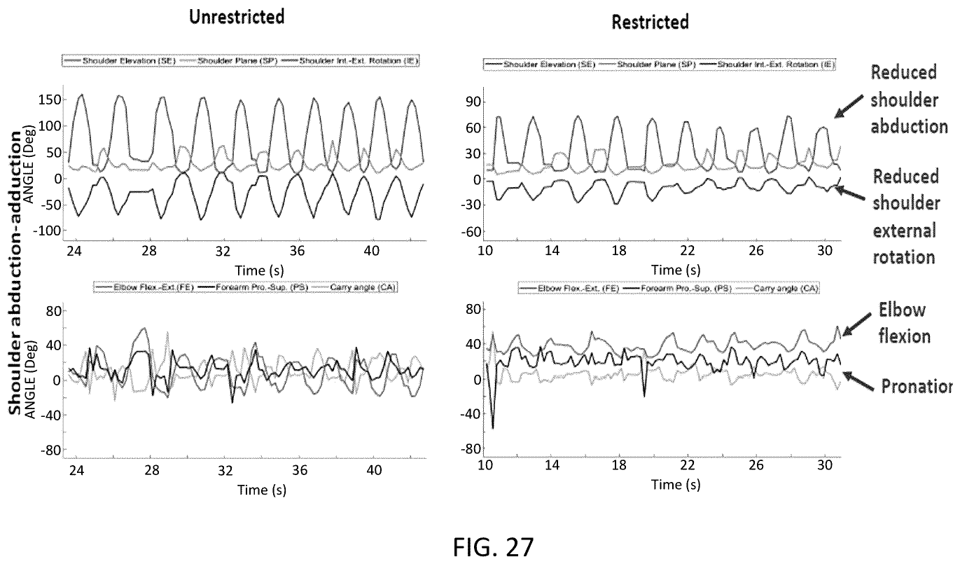

FIG. 27 shows example data comparing unrestricted and restricted joint movement. Unrestricted shoulder abduction (left panel) requires shoulder external rotation as well as forearm supination. If shoulder abduction is restricted, it is important to understand why it is so to create a rational treatment plan. Movement diagnostics can aid in understanding where (location of) the key restrictions are and what type of restrictions they are (for example, Muscle weakness or stiffness or joint restriction).

In terms of location, if only the shoulder abduction is restricted but there is full range at the other joints then that is the only movement that needs treatment. If shoulder external rotation is restricted as well, but not supination then treatment needs to be initiated for shoulder external rotation first to restore shoulder abduction. If both shoulder external rotation and supination are restricted, then the supination needs to be fixed as well. The right panel shows that the person is unable to supinate, which limits both shoulder external rotation and shoulder abduction. The quality of the movement can differentiate between a joint restriction or a muscular restriction which in combination with the location of the restriction can guide the appropriate treatment. A therapist can utilize this data to develop a rehabilitation plan. For example, after a stroke, a patient can exhibit shoulder internal rotation, elbow flexion, forearm pronation, and finger flexion. By being able to detect these occurrences and the degree at which they are occurring, a therapist can develop a recovery plan.

The data collected from the wearable inertial sensors enable therapists to visualize individual joint angles (elbow flexion, forearm pronation/supination, shoulder plane, elevation, and internal/external rotation) during complex hand movements. All 5 joint angle measurements obtained during specific hand movements form patterns, as seen in FIGS. 26 and 27. When a patient is being evaluated with WIS for such hand movements, the joint angle measurements may deviate from the normative measurements. The data acquired allows therapists and clinicians to visually inspect the data collected and observe these deviations easily and propose corrective treatment. Machine learning or other algorithmic approaches can be used automate and aid the therapists/clinicians during this identification process.

### Comparison To Existing Systems

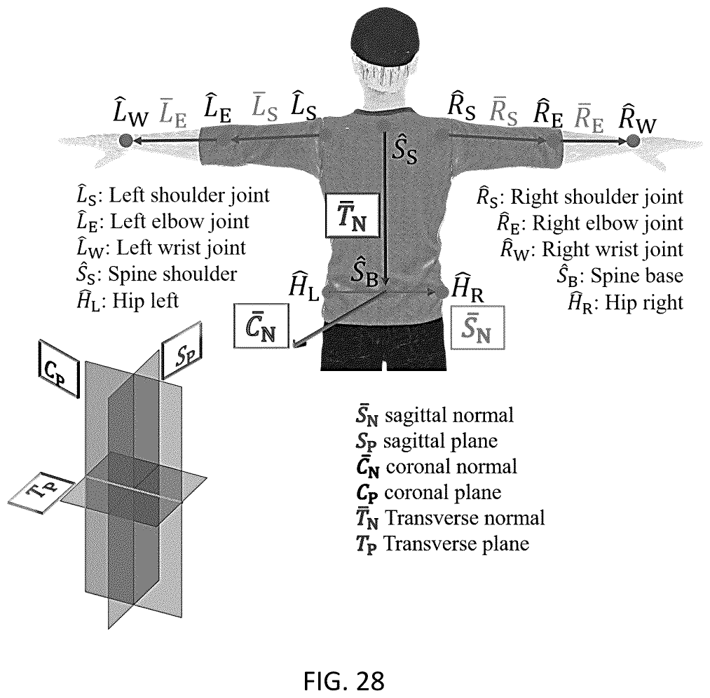

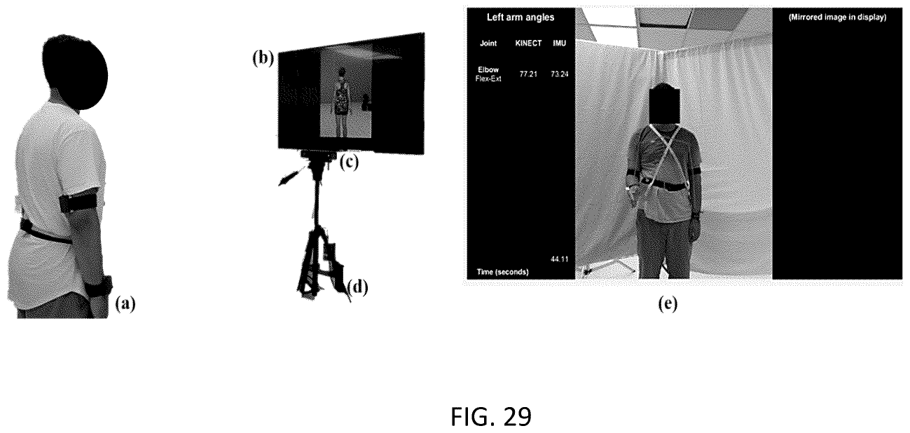

FIGS. 28, 29, 30, 31, 32 and 33 show results of an example experiment comparing system 100 to an existing system, the Microsoft Kinect. FIG. 28 shows joint positions, joint vectors, and anatomical planes used to calculate joint angles using the Kinect. FIG. 29 shows a schematic representation of subject testing (a) Subject wearing WISE modules and standing in front of the Kinect; (b) ROM exercises instructed by the virtual coach displayed on a TV screen; (c) Kinect markerless motion capture system; (d) Tripod stand for mounting Kinect; and (e) MATLAB interface for sampling data from Kinect and WISE systems.

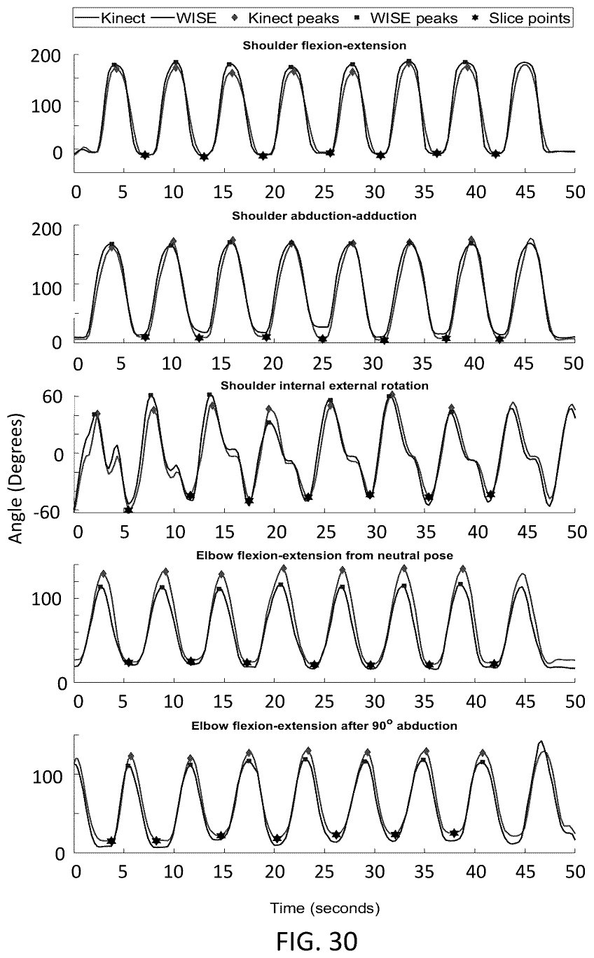

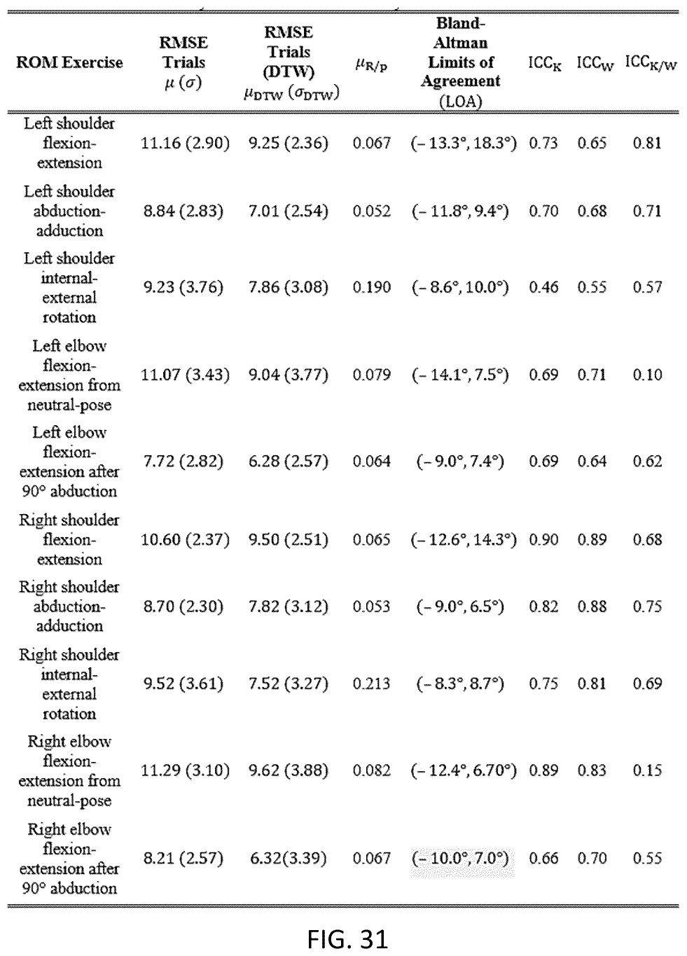

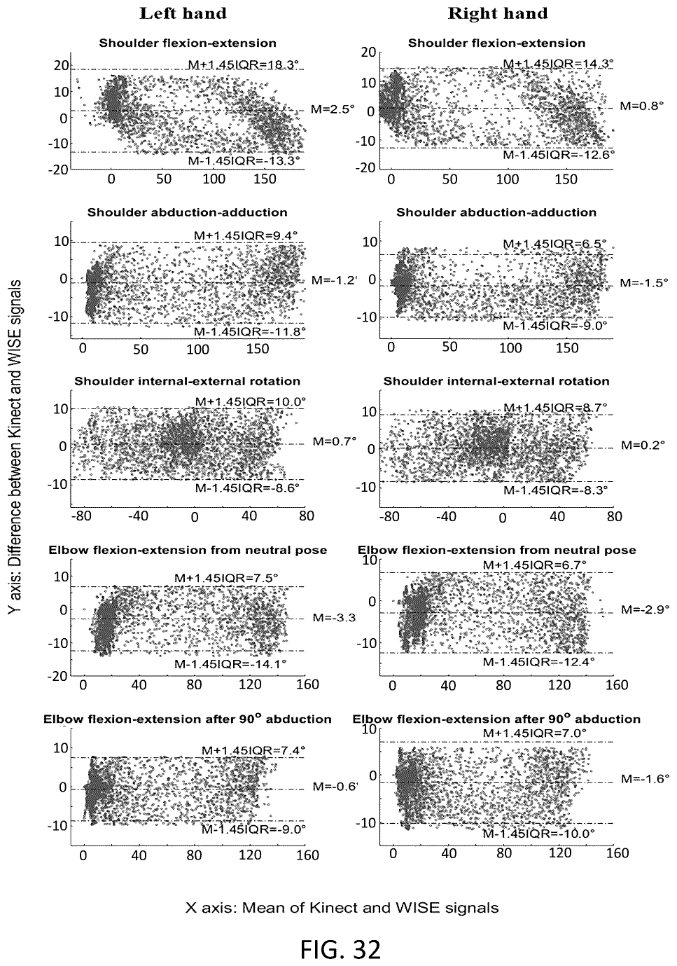

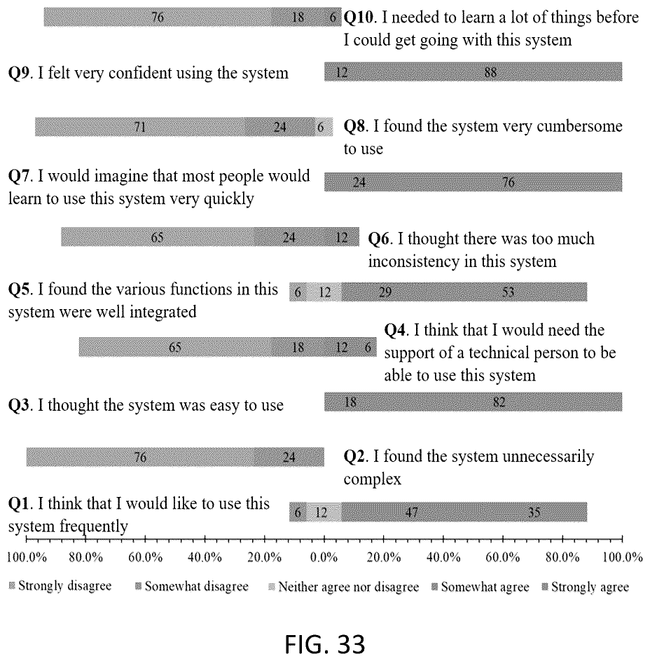

FIG. 30 shows comparative date of the Kinect and WISE system 100 data obtained from the right arm movements. FIG. 31 is a table summarizing the mean and standard deviation of the root mean square error for each trial before and after dynamic time warping between the Kinect and WISE 100 measurements, mean error relative to peak ROM, Bland-Altman limits of agreement, intra-class correlation coefficients for consistency within each measurement system and between the two systems. FIG. 32 shows Bland-Altman plots for all 10 ROM exercises. FIG. 33 shows a horizontal bar chart of responses (in percentage) of the system usability scale.

The Kinect provides a total of 25 joint coordinates of a human in 3D workspace extracted from the image and depth information However, measurements from the Kinect do not yield sufficient information to characterize movement in the JCS framework. Specifically, by using the shoulder, elbow, and wrist positions obtained from the Kinect, it is not possible to resolve the three principal axes corresponding to each of the shoulder and elbow joints. Thus, instead of using the JCS framework for computing shoulder abduction-adduction and flexion-extension, the vector projection approach was adapted.

To illustrate the JA computation from the Kinect, characters with bar accent are used to denote vectors, e.g., “F̅” and hat accent “\hat{F}” to denote coordinates of a point in the 3D space. It began by constructing a subject centric body coordinate frame for the UE, FIG. 27, by using the real-time joint positions obtained from the Kinect to compute the JA. The sagittal plane (S_{P}), coronal plane (C_{P}), and transverse plane (T_{P}) divide the human body into the left-right, frontal-rear, and top-bottom halves, respectively. The vectors S̅_{N}, C̅_{N}, and T̅_{N} represent vectors normal to the sagittal, coronal, and transverse planes, respectively. The left and right arm vectors are constructed by subtracting the proximal joint coordinate (shoulder) from the distal joint coordinate (elbow) as below.

F̅_{S}=\hat{F}_{E}−\hat{F}_{S}, (27)

The shoulder joint angles are defined as follows: (i) shoulder flexion-extension (θ_{FE}) is the movement of the arm vector F̅_{S} in a plane parallel to the sagittal plane and (ii) shoulder abduction-adduction (θ_{BD}) is the movement of the arm vector in a plane parallel to the coronal plane. The angle calculation for these movements of the left shoulder are performed as below.

θ_{LFE}=atan2(−L̅_{S}·C̅_{N}, L̅_{S}·T̅_{N}) (28)

θ_{LBD}=atan2(−L̅_{S}·S̅_{N}, L̅_{S}·T̅_{N}) (29)

The forearm vectors are constructed from the joint coordinates of elbows and wrists. The left elbow flexion-extension α_{LFE} angle computation is performed as below.

α_{LFE}=cps⁻¹(L̅_{S}·L̅_{E}) (30)

Note that the forearm pronation-supination angle cannot be computed from the Kinect data due to the lack of sufficient information.

The shoulder internal-external rotation (θ_{IE}) of the left or right arm cannot be computed with the information of shoulder vectors L̅_{S} and L̅_{R}. Hence, the information of the elbow vectors L̅_{E} and R̅_{E} was utilized to compute this angle by projecting it on the transverse plane as below.

θ_{LIE}=atan2(C̅_{N}·L̅_{E}, S̅_{N}·L̅_{E}), α_{LFE}≥30° (31)

Thus, the shoulder internal-external rotation calculation works only if the elbow is flexed beyond 30°. Finally, the computation for the right side of the body also utilizes similar equations as above and care is taken to ensure that extension, adduction, and external rotation are denoted as (−). FIG. 27 illustrates all the UE joint coordinates and vectors used for the calculation of JA with the Kinect data.

As delineated above, unlike with the WISE system 100, measurements from the Kinect cannot use the JCS framework. Thus, the WISE system 100 is modified to facilitate one-on-one comparison with the Kinect-based measurements of the computation of shoulder flexion-extension and abduction-adduction . The modified approach is outlined below.

The principal axes of the WISE module B (X_{B}−Y _{B}−Z _{B}) are used to recreate the transverse, sagittal, and coronal planes on the human body such that the X_{B}, Y_{B}, and Z_{B} axes are normal to the transverse, sagittal, and coronal planes, respectively. The (X_{B}, Y_{B}, Z_{B}) axes represented in the sensor coordinate frame [symbol] _{S} are transformed to (\tilde{X}_{B}, \tilde{Y}_{B}, \tilde{Z}_{B}) in [symbol] _{W}. The shoulder flexion-extension angle θ_{FE} and abduction-adduction angle θ_{BD} are computed for the left arm as below.

θ_{LFE}=atan2(Y_{LA}·\tilde{Z}_{B}, Y_{LA}·\tilde{X}_{B}) (32)

θ_{LBD}=atan2(Y_{LA}·\tilde{Z}_{B}, Z_{LA}·\tilde{X}_{B} (23)

The angles for the right side are computed so that the signs of flexion and abduction are (+).

Seventeen healthy subjects (11 male and 6 female) were recruited for the study in the following age groups: 18-24 years (n=8), 25-34 years (n=6), 35-44 years (n=1), 45-54 years (n=1), and 55-64 years (n=1). The participants wore the WISE modules and stood six feet in front of the Kinect. Video tutorials recorded with a 3D animated human model served as the virtual coach displayed on a television screen. The virtual coach demonstrated each exercise first and then instructed the subjects to perform the demonstrated exercises along with the coach for eight trials of the ten ROM exercises. At the end of the session, subjects were presented with a SUS questionnaire that included ten questions (with five positive and five negative statements). The questionnaire sought respondents' opinion about using the virtual coach for ROM exercises on a five-point Likert scale.

Using MATLAB version 2019a (MathWorks, Inc., Natick, MA), a routine was created for real-time data acquisition to compare the WISE and Kinect measurements. The experimental setup is illustrated in FIG. 28. Three user interfaces were created: (i) a video display for subjects to view the virtual coach (FIG. 28b); (ii) Kinect's video stream superimposed with numerical values of the joint angles computed from the Kinect and WISE systems (FIG. 28e); and (iii) animated plots for JA visualization from the Kinect and WISE systems. Each subject performed the ROM exercises in the following sequence: (i) left shoulder flexion-extension (θ_{LFE}), (ii) left shoulder abduction-adduction (θ_{LBD}), (iii) left elbow flexion-extension from neutral-pose (α_{LFE}_{₁}), (iv) left elbow flexion-extension with 90° shoulder abduction (α_{LFE}_{₂}), (v) left shoulder internal-external rotation with 90° elbow flexion (θ_{LIE}), (vi) right shoulder flexion-extension (θ_{RFE}), (vii) right shoulder abduction-adduction (θ_{RBD}), (viii) right elbow flexion-extension from neutral-pose (α_{RFE}_{₁}), (ix) right elbow flexion-extension with 90° shoulder abduction (α_{RFE}_{₁}), and (x) right shoulder internal-external rotation with 90° elbow flexion (θ_{RIE}). The complete procedure lasted approximately 20 minutes for each subject. The JA data computed from the Kinect and WISE systems were saved in a text file for post-processing.

The data was analyzed using MATLAB with the command f indpeaks to determine the peak ROM measured using the Kinect (p_{ROM}_{_{K}}^{E}) and WISE (p_{ROM}_{_{W}}^{E}) systems, where superscript “E” represents specific ROM exercises. The temporal signal for each movement was then spliced to separate the trials (see FIG. 30). The ROM from the two systems were compared by determining the RMSE (R_{S}_{ᵢ}^{E}) for each trial, where subscripts “s” and “i” represent the subject number and trial number, respectively. The extracted peaks and RMSE were used to compute the mean error relative to the peak ROM

$$( {μ}_{R / p} = \frac{1}{n} {\sum }_{i = 1}^{n} \frac{{R}_{{S}_{i}}^{E}}{{( {p}_{{ROM}_{K}}^{E} )}_{i}} , where n = {6}_{trials} \times {17}_{subjects} ) ,$$

as well as the mean (ν) and standard deviation (σ) of the RMSE of trials two to seven across all subjects.

Data acquisition in the MATLAB environment used a line-by-line program execution which caused a systematic lag/lead between the Kinect and WISE measurements. To mitigate the temporal error, dynamic time warping (DTW) was applied to the Kinect and WISE measurements using the MATLAB command dtw similar to the procedure outlined in. Prior to applying DTW, the time series joint angle signals were scaled to [−1,1] by using the corresponding peak values for the Kinect and WISE measurements. Following the application of DTW, the data were rescaled by the peak values used previously for scaling. The peak and RMSE calculations described above were repeated using the joint angle output of DTW to compute the mean (μ_{DTW}) and standard deviation (σ_{DTW}) of the RMSE for each trial.

The Bland-Altman statistic establishes the agreement between two measurement systems by computing the limits of agreement (LOA). Following the application of DTW, the error signal between the Kinect and WISE systems exhibited high kurtosis and skewness indicating a non-normal distribution. Thus, Kimber's outlier rejection technique was applied to reject outlier data points beyond (Q₁−γ(M−Q₁), Q₃+γ(Q₃−M)), where Q₁, Q₃, and M denote the first quartile, third quartile, and median, respectively. The multiplier γ=1.5 is a commonly used parameter for outlier rejection, thus it was used for rejecting the outliers in the Kinect and WISE signals. The outputs of the outlier rejection procedure also exhibited non-normal distributions in the error signal. Thus, the Bland-Altman test for non-parametric signals, which defines the LOA as the median ±1.45 times the interquartile range (i.e., M±1.45×IQR), was applied to the remainder of the Kinect and WISE data after the outlier rejection. The peaks obtained from the Kinect and WISE measurements after DTW were also used to compute the intra-class correlation coefficients (ICC). Two methods were used to determine (i) the test-retest consistency (ICC(C,1)) of the Kinect (ICC_{K}) and WISE (ICC_{W}) between trials and (ii) the absolute agreement (ICC(A,1)) between the measurements of ROM peaks of the Kinect and WISE (ICC _{K/W}). The SUS responses for ten questions obtained from the subjects were analyzed for reliability using the Cronbach's alpha and the final SUS score was computed.

The mean and standard deviations of the RMSE before and after correction for temporal delays using DTW between the joint angle measurements obtained from the Kinect and WISE systems for 17 subjects and 10 ROM exercises, Bland-Altman LOA, intra-class correlation coefficients for each device (ICC_{K}, ICC_{W}), and the absolute agreement between the two devices (ICC_{K/W}) are shown in FIG. 31. The mean of the RMSE shows that the ROM measurements in the coronal, transverse, and sagittal planes had errors of less than 9°, 10°, and 12°, respectively. After the application of DTW, the mean RMSE errors in the coronal, transverse, and sagittal planes decreased to less than 8°, 8°, and 10°, respectively. The Bland-Altman LOA, with 95% confidence intervals, for ROM measurements in the coronal, transverse, and sagittal planes were determined to be in the range of (−12.0°, 10.0°), (−9.5°, 10°), and (−14.0°, 18.0°), respectively. The intra-class correlation coefficients for the Kinect (ICC_{K}) and WISE (ICC_{W}) systems indicated very good repeatability within each system especially for right arm movements and slightly lower for left arm movements. The ICC was lowest for the left shoulder internal-external rotation. The ICC_{K/W}, which compared the consistency between the Kinect and the WISE systems, showed moderate to very good agreement for all movements except the left and right elbow flexion-extension from the neutral-pose.

The Bland-Altman plots (i.e., mean versus difference) for the DTW-processed signals of the Kinect and WISE systems for the 10 ROM exercises are presented in FIG. 32. The measurements for coronal plane ROM exercises (i) left and right elbow flexion-extension with 90° shoulder abduction and (ii) right shoulder abduction-adduction produced Bland-Altman LOA within ±10°. However, for left shoulder abduction-adduction measurements the LOA was (−12.0°, 9.4°), which is slightly higher than the ±10° range. The transverse plane ROM exercises for the left and right shoulder internal-external rotation with 90° elbow flexion were also within the ±10° acceptance limits.

The SUS response percentages are plotted in a bar chart as shown in FIG. 33. The Cronbach's alpha for the five-point SUS scale for the ten questions was computed to be above 0.7, indicating acceptable reliability. A majority of the users found the virtual coach-based ROM tutoring system to be easy to use (82% strongly agreed) and well-integrated (82% agreed or strongly agreed) and reported that they would be confident in using it (88% strongly agreed) and would like to use it (82% agreed or strongly agreed). Overall, the SUS score showed relatively high third and first quartile scores of 97.5 and 82.5, respectively, with the interquartile range of 15 and the minimum score of 65, suggesting that the subjects were interested in using the animated virtual coach for the guided ROM exercises.

In conclusion, the results show moderate to very good within-device agreement for each of the measurement systems. The discrepancy between the two devices was within ±10° for most of the ROM exercises. The between-device agreement was moderate to very good in the coronal and transverse planes for the following ROM exercises: (i) shoulder abduction-adduction, (ii) elbow flexion-extension with the shoulder abducted at 90°, and (iii) shoulder internal-external rotation. Even though, there are no quantified clinical acceptance limits for the ROM assessment, prior literature has suggested ±10° as acceptable LOA for the Bland-Altman statistic. Furthermore, the RMSE and Bland-Altman LOA results suggest the concurrence between the two measurement systems was best in the coronal plane. However, the RMSE for exercises in the sagittal plane, i.e., (i) elbow flexion-extension from neutral-pose and (ii) shoulder flexion-extension showed greater discrepancy between the two devices. These discrepancies persisted despite the adaptation of the Kinect-based vector projection method for computing joint angles with the WISE system. This can be explained by the problem of joint occlusion during movements in the sagittal plane when the Kinect is placed in front of the subject. Alternatively, when the elbow flexion-extension exercise was performed with the shoulder abducted at 90°, the occlusion did not occur and resulted in the least discrepancy between the two systems. Next, the ROM exercises for internal-external rotation in the transverse plane were modified by the introduction of 90° elbow flexion, which enabled the use of elbow vector obtained from the Kinect measurements for the computation of shoulder internal-external rotation angle. Furthermore, the forearm pronation-supination angles could not be computed from the Kinect measurements. Although Kinect has been used extensively for exergames and rehabilitation, above results suggest that joint angle measurements from such markerless motion capture devices lack the ability to resolve motion in three planes of movement for each joint (i.e., shoulder and elbow) as required by the JCS framework. In contrast, the WISE system provides a robust integration of measurements from multiple wearable sensors for the shoulder and elbow joints allowing continuous real-time measurements of joint angles in three planes for each joint. Finally, the SUS scores suggest that subjects were interested in using the animated virtual coach for the guided ROM exercises.

In a prior study, a method was developed to remotely assess grasping performance using real-time data in patients with multiple sclerosis. Using the WISE system, a similar telerehabilitation intervention can be developed for patients in need of upper extremity ROM assessment and rehabilitation exercises. In such a scenario, the therapists can use the virtual coach to provide an individualized battery of exercises, enabling patients to perform exercises at home while asynchronous measurements using the WISE platform can capture and transmit the information to the therapist. The granular ROM data in all three planes can be useful to bridge the gap between laboratory research on motion analysis and translation to clinical practice. Although the current implementation of the WISE system was restricted to a computer-based interface, in prior work feasibility of interfacing medical devices with smartphones was demonstrated. In a similar vein, the WISE system can be interfaced to mobile devices such as tablets and smartphones to render a portable mHealth system. Future work will consider the use of the WISE virtual coach and a guided mounting interface for the sensors, as well as feedback systems that enable the virtual coach to tailor exercises based on the data acquired from the sensors.

### Conclusion

Disclosed is a mechatronic approach to design and develop a WIS system for tri-planar upper extremity ROM assessment. Two software-based signal processing methods were introduced to correct the orientation misalignment between the sensor and its housing. The WIS module measurements were benchmarked against a goniometer on a turntable for repeated measurements and the results show acceptable agreement between measurements in all axes. Furthermore, the experimental measurements were analyzed for accuracy and reliability, and indicate acceptable tolerance limits for rehabilitative applications. Next, the clinically accepted JCS-based ROM assessment technique was integrated with the WIS system for ease of use by rehabilitation clinicians and translation to clinical practice. The results illustrate simultaneous availability of all joint angles to enable clinicians to identify movement restrictions accurately and tailor treatment effectively.

There are several limitations to the work presented. First, in-house desktop milling machines were used to machine the WIS module PCBs, yielding a quick turnaround time but a large manufacturing footprint. Second, currently the software signal processing, data acquisition, and data analysis algorithms are all implemented using MATLAB, which is unsuitable for translation of the WIS system to patients' homes and clinical practice. The feasibility of using the WIS system under the JCS framework for ROM assessment was examined with a single healthy subject.

Future work will address several of the aforementioned limitations. By leveraging state-of-the-art manufacturing capabilities, the PCB design of the WIS module can be reduced in size, improving its form factor, comfort level, and wearability. A formal study on user experience will be conducted related to such an updated WIS module design. An exergame framework integrates the WIS system in the open-source Unity3D environment and eliminate the need for commercial software tools. The exergame environment includes two human models (i) an animated virtual coach to instruct the users for performing ROM exercises and (ii) a patient model that simulates the user's movements retrieved from the sensor measurements. An instructor interface enables intuitive visualization and comparison between the animated virtual coach instruction vs. the patient ROM data to facilitate patient performance assessment and feedback. With such an interface, therapists and clinicians will be able to tailor individualized treatment for the patients.

In prior research, the use of BLE-based devices for interfacing with smartphone applications was demonstrated. In a similar vein, Unity3D-based applications are compatible for deployment on smartphone interfaces to facilitate the development of smartphone connected WIS modules for patient rehabilitation and ROM assessment. In prior research, the ability to utilize mechatronic approaches was also demonstrated for creating low-cost, reproducible, prototypes of a grasp rehabilitator. In a related study, six copies of the grasp rehabilitator were reproduced and utilized within a telemedicine framework to remotely assess the grasp performance and therapy compliance in patients with multiple sclerosis. In future work, a similar approach will be adopted to use small-footprint, reproduced versions of WIS modules for ROM assessment of patients in clinical and telemedicine settings to generate clinically relevant efficacy, validation, and compliance data for these devices.

The following references are incorporated by reference in their entirety:
A. I. Cuesta-Vargas, A. Galán-Mercant, and J. M. Williams, “The use of inertial sensors system for human motion analysis,” Phys. Therapy Rev., vol. 15, no. 6, pp. 462-473, Dec. 2010.
A. Mathis et al., “DeepLabCut: Markerless pose estimation of userdefined body parts with deep learning,” Nature Neurosci., vol. 21, no. 9, pp. 1281-1289, Sep. 2018.
A. RajKumar et al., “Usability study of wearable inertial sensors for exergames (WISE) for movements assessment and exercise,” mHealth, (under rev.).
A. Rajkumar et al., “Wearable inertial sensors for range of motion assessment,” IEEE Sens. J., doi: 10.1109/JSEN.2019.2960320, 2019.
A. Rajkumar, C. Arora, B. Katz, and V. Kapila, “Wearable smart glasses for assessment of eye-contact behavior in children with autism,” in Proc. Design Med. Devices Conf., Apr. 2019, p. V001T09A006.
A. RajKumar, S. Bilaloglu, P. Raghavan, and V. Kapila, “Grasp rehabilitator: A mechatronic approach,” in Proc. Design Med. Devices Conf., Apr. 2019, p. V001T03A007.
A. F. De Winter et al., “Inter-observer reproducibility of measurements of range of motion in patients with shoulder pain using a digital inclinometer,” BMC Musculoskelet. Disord., vol. 5, no. 18, pp. 1-8, 2004.
Benjamin E J, Muntner P, Alonso A, et al. Heart disease and stroke statistics-2019 update: A report from the American Heart Association. Circulation. 2019;139:e56-528.
Bland J M, Altman D G. Statistical methods for assessing agreement between two methods of clinical measurement. International Journal of Nursing Studies. 2010;47:931-6.
Bonnechere B, Jansen B, Salvia P, et al. Validity and reliability of the Kinect within functional assessment activities: Comparison with standard stereophotogrammetry. Gait and Posture. 2014;39:593-8.
Bonnechere B, Jansen B, Salvia P, et al. What are the current limits of the Kinect sensor? In: Ninth International conference on Disability, Virtual Reality and Associated Technologies. 2012. p. 287-94.
Bosch Sensortech GmbH. (2016). BNO055 Intelligent 9-Axis Absolute Orientation Sensor. Accessed: Jul. 8,2019. [Online]. Available: https://www.bosch-sensortec.com
Brooke J. SUS-A quick and dirty usability scale. Usability Evaluation in Industry. 1996;189:4-7.
Burdea G. Virtual rehabilitation—Benefits and challenges. Methods of Information in Medicine. 2003;42:519-23.
Caggianese G, Cuomo S, Esposito M, et al. Serious games and in-cloud data analytics for the virtualization and personalization of rehabilitation treatments. IEEE Transactions on Industrial Informatics. 2019;15:517-26.
Choppin S, Lane B, Wheat J. The accuracy of the Microsoft Kinect in joint angle measurement. Sports Technology. 2014;7:98-105.
Cronbach L J. Coefficient alpha and the internal structure of tests. Psychometrika. 1951;16:297-334.
D. D. Hardwick and C. E. Lang, “Scapular and humeral movement patterns of people with stroke during range-of-motion exercises,” J. Neurol. Phys. Therapy, vol. 35, no. 1, pp. 18-25, March 2011.
D. H. Gates, L. S. Walters, J. Cowley, J. M. Wilken, and L. Resnik, “Range of motion requirements for upper—limb activities of daily living,” Amer. J. Occupational Therapy, vol. 70, no. 1, pp. 7001350010-1-7001350010-10, January/February 2016.
De Winter A F, Heemskerk M A, Terwee C B, et al. Inter-observer reproducibility of measurements of range of motion in patients with shoulder pain using a digital inclinometer. BMC Musculoskeletal Disorders. 2004;5:1-8.
E. Bachmann, I. Duman, U. Usta, R. Mcghee, X. Yun, and M. Zyda, “Orientation tracking for humans and robots using inertial sensors,” in Proc. IEEE Int. Symp. Comput. Intel!. Robot. Automat. (CIRA), Nov. 1999, pp. 187-194.
E. Knippenberg, J. Verbrugghe, I. Lamers, S. Palmaers, A. Timmermans, and A. Spooren, “Markerless motion capture systems as training device in neurological rehabilitation: A systematic review of their use, application, target population and efficacy,” J. Neuroeng. Rehabil., vol. 14, no. 1, pp. 1-11, December 2017.
E. Palermo, S. Rossi, F. Marini, F. Patane, and P. Cappa, “Experimental evaluation of accuracy and repeatability of a novel body-to-sensor calibration procedure for inertial sensor-based gait analysis,” Measurement, vol. 52, pp. 145-155, June 2014.
Edwards J. Wireless sensors relay medical insight to patients and caregivers. IEEE Signal Processing Magazine. 2012;29:8-12.
Feinberg C, Shaw M, Palmeri M, et al. Remotely supervised transcranial direct current stimulation (RS-tDCS) paired with a hand exercise program to improve manual dexterity in progressive multiple sclerosis: A randomized sham controlled trial. Neurology. 2019;92:P5.6-009.
Fernandez-Baena A, Susin A, Lligadas X. Biomechanical validation of upper-body and lower-body joint movements of Kinect motion capture data for rehabilitation treatments. In: Fourth International Conference on Intelligent Networking and Collaborative Systems. 2012. p. 656-61.
G. Paraskevas, A. Papadopoulos, B. Papaziogas, S. Spanidou, H. Argiriadou, and J. Gigis, “Study of the carrying angle of the human elbow joint in full extension: A morphometric analysis,” Surgical Radio!. Anatomy, vol. 26, no. 1, pp. 19-23, February 2004.
G. Wu et al., “ISB recommendation on definitions of joint coordinate system of various joints for the reporting of human joint motion—Part I: Ankle, hip, and spine,” J. Biomech., vol. 35, no. 4, pp. 543-548, April 2002.
G. Wu et al., “ISB recommendation on definitions of joint coordinate systems of various joints for the reporting of human joint motion—Part II: Shoulder, elbow, wrist and hand,” J. Biomech., vol. 38, no. 5, pp. 981-992, May 2005.
GmbH BS. BNO055 Intelligent 9-axis Absolute Orientation Sensor [Internet]. https://cdn-shop.adafruit.com/datasheets/BST_BNO055_DS000_12.pdf. 2016.
Hawi N, Liodakis E, Suero EM, et al. Range of motion assessment of the shoulder and elbow joints using a motion sensing input device: A pilot study. Technology and Health Care. 2014;22:289-95.
Huber ME, Seitz AL, Leeser M, et al. Validity and reliability of Kinect skeleton for measuring shoulder joint angles: A feasibility study. Physiotherapy. 2015;101:389-93.
I. H. Lopez-Nava and A. Munoz-Melendez, “Wearable inertial sensors for human motion analysis: A review,” IEEE Sensors J., vol. 16, no. 22, pp. 7821-7834, November 2016.
J. Diebel, “Representing attitude: Euler angles, unit quaternions, and rotation vectors,” Stanford Univ., Stanford, CA, USA, Tech. Rep., 2006.
J. Fei and X. Liang, “Adaptive backstepping fuzzy neural network fractional-order control of microgyroscope using a nonsingular terminal sliding mode controller,” Complexity, vol. 2018, June 2018, Art. no. 5246074.
J. Fei and Z. Feng, “Adaptive fuzzy super-twisting sliding mode control for microgyroscope,” Complexity, vol. 2019, February 2019, Art. no. 6942642.
J. Lv, A. A. Ravankar, Y. Kobayashi, and T. Emaru, “A method of lowcost IMU calibration and alignment,” in Proc. IEEE/SICE Int. Symp. Syst. lntegr. (SII), December 2016, pp. 373-378.
J. Macdermid, K. Sinden, T. Jenkyn, G. Athwal, T. Birmingham, and L. Khadilkar, “An analysis of functional shoulder movements during task performance using Dartfish movement analysis software,” Int. J. Shoulder Surg., vol. 8, no. 1, pp. 1-9, January-March 2014.
J. Rohac, M. Sipos, and J. Simanek, “Calibration of low-cost triaxial inertial sensors,” IEEE Instrum. Meas. Mag., vol. 18, no. 6, pp. 32-38, December 2015.
J. Vc\check{ }elák, P. Ripka, J. Kubik, A. Platil, and P. KaSpar, “AMR navigation systems and methods of their calibration,” Sens. Actuators A, Phys., vols. 123-124, pp. 122-128, September 2005.
Jonsdottir J, Bertoni R, Lawo M, et al. Serious games for arm rehabilitation of persons with multiple sclerosis. A randomized controlled pilot study. Multiple Sclerosis and Related Disorders. 2018;19:25-9.
K. N. An, B. F. Morrey, and E. Y. S. Chao, “Carrying angle of the human elbow joint,” J. Orthopaedic Res., vol. 1, no. 4, pp. 369-378,1983.
K. E. Ravenek et al., “A scoping review of video gaming in rehabilitation,” Disabil. Rehabil. Assist. Technol., vol. 11, no. 6, pp. 445-453,2016.
Kim J, Campbell AS, de Avila BEF, et al. Wearable biosensors for healthcare monitoring. Nature Biotechnology. 2019;37:389-406.
Kimber AC. Exploratory data analysis for possibly censored data from skewed distributions. Journal of the Royal Statistical Society. 1990;39:21-30.
Kitsunezaki N, Adachi E, Masuda T, et al. Kinect applications for the physical rehabilitation. In: IEEE International Symposium on Medical Measurements and Applications. 2013. p. 294-9.
Kurillo G, Chen A, Bajcsy R, et al. Evaluation of upper extremity reachable workspace using Kinect camera. Technology and Health Care. 2013;21:641-56.
Kurillo G, Han JJ, Obdržálek Š, et al. Upper extremity reachable workspace evaluation with Kinect. In: Studies in Health Technology and Informatics. 2013. p. 247-53.
M. Andriluka, L. Pishchulin, P. Gehler, and B. Schiele, “2D human pose estimation: New benchmark and state of the art analysis,” in Proc. IEEE Conf. Comput. Vis. Pattern Recognit., June 2014, pp. 3686-3693.
M. EI-Gohary and J. McNames, “Shoulder and elbow joint angle tracking with inertial sensors,” IEEE Trans. Biomed. Eng., vol. 59, no. 9, pp. 2635-2641, June 2012.
M. losa, P. Picerno, S. Paolucci, and G. Morone, “Wearable inertial sensors for human movement analysis,” Expert Rev. Med. Devices, vol. 13, no. 7, pp. 641-659, April 2016.
M. Kim and D. Lee, “Wearable inertial sensor based parametric calibration of lower-limb kinematics,” Sens. Actuators A, Phys., vol. 265, pp. 280-296, October 2017.
M. Malik et al., “Upper extremity telerehabilitation for progressive multiple sclerosis,” Neurology, submitted for publication.
M. S. Karunarathne, S. Li, S. W. Ekanayake, and P. N. Pathirana, “An adaptive orientation misalignment calibration method for shoulder movements using inertial sensors: A feasibility study,” in Proc. Int. Symp. Bioelectron. Bioinform. (ISBB), October 2015, pp. 99-102.
M. Windolf, N. Gëtzen, and M. Morlock, “Systematic accuracy and precision analysis of video motion capturing systems—Exemplified on the Vicon-460 system,” J. Biomech., vol. 41, no. 12, pp. 2776-2780, Augugust 2008.
McGraw K O, Wong S P. Forming inferences about some intraclass correlation coefficients. Psychological Methods. 1996;1:30-46.
Nordic Semiconductors. Gazell Link Layer User Guide. Accessed: Jul. 8, 2019. [Online]. Available: http://www.infocenter.nordicsemi.com/Pfister A, West AM, Bronner S, et al. Comparative abilities of Microsoft Kinect and Vicon 3D motion capture for gait analysis. Journal of Medical Engineering and Technology. 2014;38:274-80
R. L. Gajdosik and R. W. Bohannon, “Clinical measurement of range of motion: Review of goniometry emphasizing reliability and validity,” Phys. Therapy, vol. 67, no. 12, pp. 1867-1872, December 1987.
S. Bonnet, C. Bassompierre, C. Godin, S. Lesecq, and A. Barraud, “Calibration methods for inertial and magnetic sensors,” Sens. Actuators A, Phys., vol. 156, no. 2, pp. 302-311, December 2009.
S. H. Lee, C. Yoon, S. G. Chung, H. C. Kim, Y. Kwak, H.-W. Park, and K. Kim, “Measurement of shoulder range of motion in patients with adhesive capsulitis using a Kinect,” PLoS ONE, vol. 10, no. 6, June 2015, Art. no. e0129398.
S. O. H. Madgwick, A. J. L. Harrison, and R. Vaidyanathan, “Estimation of IMU and MARG orientation using a gradient descent algorithm,” in Proc. IEEE Int. Conf. Rehabil. Robot., June 2011, pp. 1-7.
S. Patel et al., “A review of wearable sensors and systems with application in rehabilitation,” J. Neuroeng. Rehabil., vol. 9, no. 1, p. 21,2012.
Seibi C, Sakoe H. Dynamic programming algorithm optimization for spoken word recognition. IEEE Transactions on Acoustics, Speech, and Signal Processing. 1978;26:43.
Sin H, Lee G. Additional virtual reality training using Xbox Kinect in stroke survivors with hemiplegia. American Journal of Physical Medicine and Rehabilitation. 2013;92:871-80.
Spong M, Hutchinson S, Vidyasagar M. Robot Modeling and Control. John Wiley and Sons, Inc, Hoboken, 2006.
Terven J R, Córdova-Esparza D M. King. A Kinect 2 toolbox for MATLAB. Science of Computer Programming. 2016;130:97-106.
Van Vuuren S, Cherney LR. A virtual therapist for speech and language therapy. In: Intelligent Virtual Agents. 2014. p. 438-48.
Vulpi F, RajKumar A, Bethi S R, et al. Signal processing and validation of orientation data from wearable inertial sensors. In: IEEE Signal Processing in Medicine and Biology. 2019. Under Review.
Wessa P. Cronbach Alpha (v1.0.5) in Free Statistics Software (v1.2.1) [Internet] https://www.wessa.net/rwasp_cronbach.wasp/.2017.
X. Chen, “Human motion analysis with wearable inertial sensors,” Ph.D. dissertation, Univ. Tennessee, Knoxville, TN, USA, 2013.
Y. Fang, J. Fei, and Y. Yang, “Adaptive backstepping design of a microgyroscope,” Micromachines, vol. 9, no. 7, p. 338, July 2018
Y.-L. Hsu et al., “A wearable inertial-sensing-based body sensor network for shoulder range of motion assessment,” in Proc. Int. Conf. Orange Technol., March 2013, pp. 328-331.
Z. Lin, Y. Xiong, H. Dai, and X. Xia, “An experimental performance evaluation of the orientation accuracy of four nine—axis MEMS motion sensors,” in Proc. 5th Int. Conf. Enterprise Syst. (ES), September 2017, pp. 185-189.

### Simulated Disability and Stroke Rehabilitation Study

The wearable inertial sensor for exergame (WISE) system facilitates real-time performance and visualization of rehabilitation exercises. When used as a tele-rehabilitation system, it permits therapists to review key performance data from a rehabilitation session with a playback interface and suggests modified or additional exercises with an instructor interface.

The system has been utilized to generate real-world data with subjects performing prescribed activities with an “active” range of motion (ROM) and with a “restricted” ROM. The following study illustrates a use case wherein a therapist or clinician can direct a user to perform prescribed activities to obtain ROM data for multiple joints. The data can then be analyzed to identify specific restrictions in various joints experienced by the user, e.g., shoulder restrictions, elbow restrictions, and forearm restrictions.

10 male and 10 female subjects were instructed to perform five exercises without restrictions, namely: shoulder flexion/extension, shoulder abduction/adduction, shoulder internal/external rotation, elbow flexion/extension, and forearm pronation/supination. Next, they were instructed to repeat these same exercises with (a) shoulder restriction; (b) elbow restriction; and (c) forearm restriction. In each case, they wore braces that introduced the required restrictions.

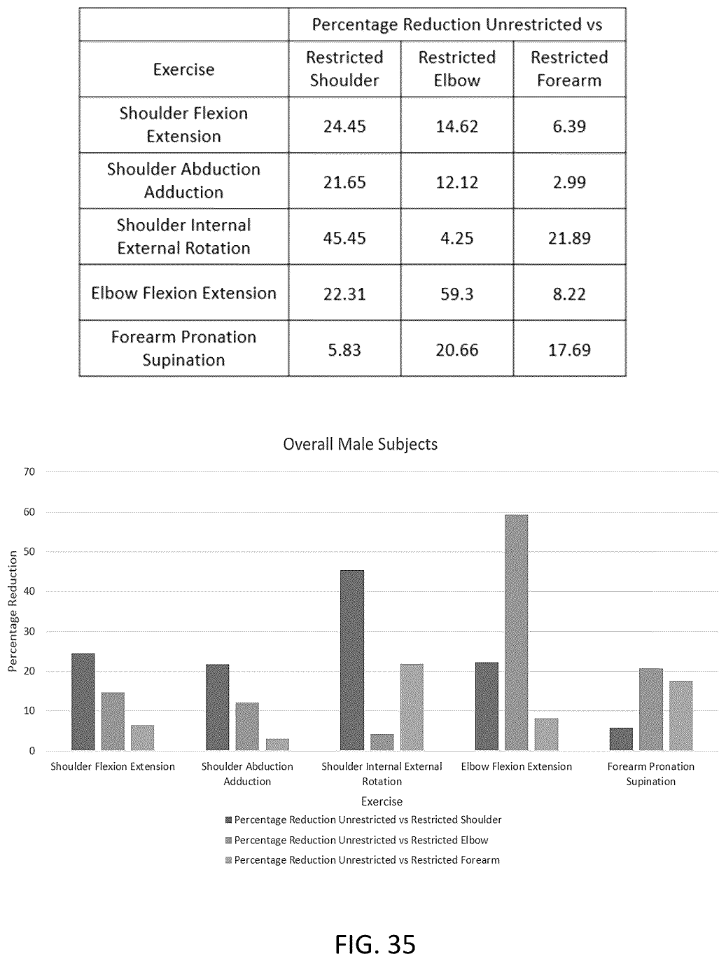

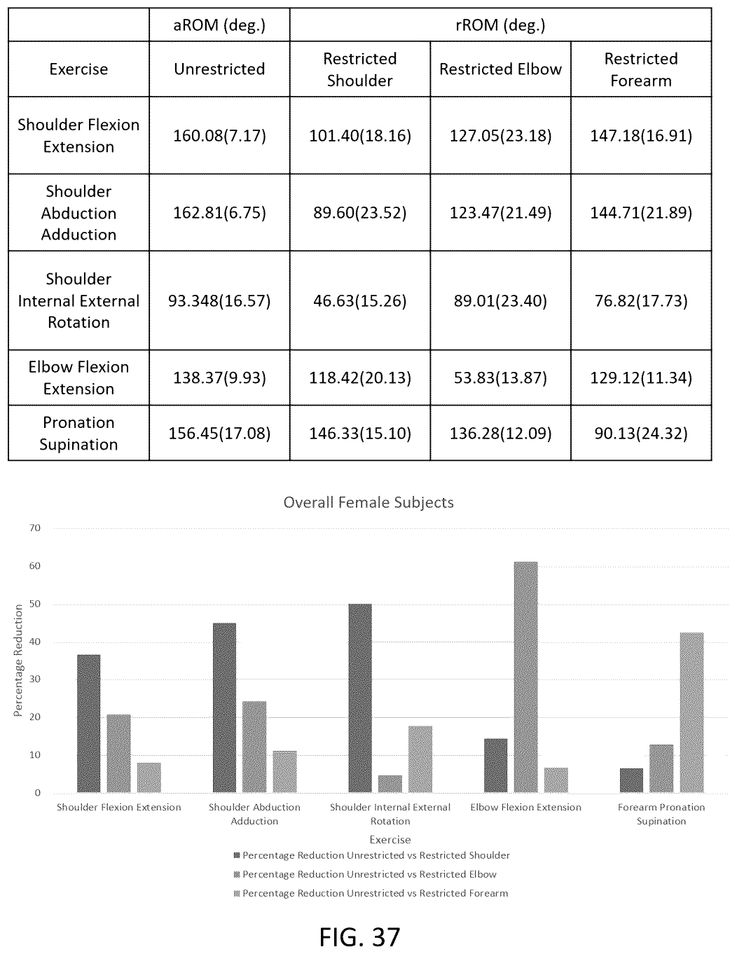

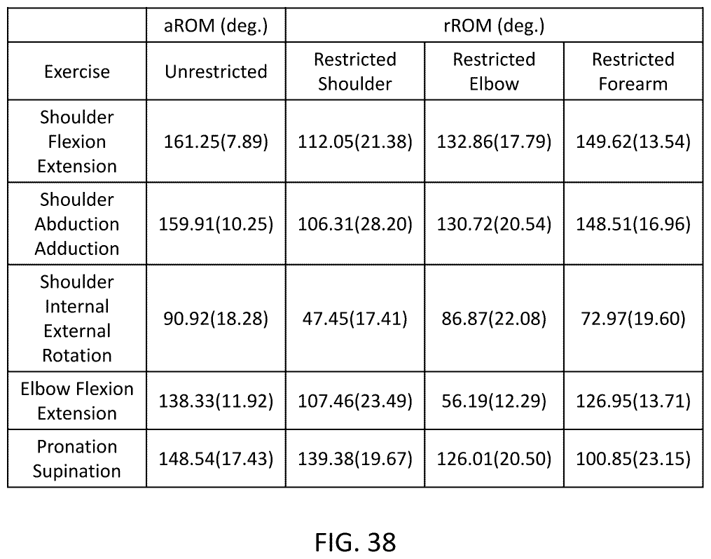

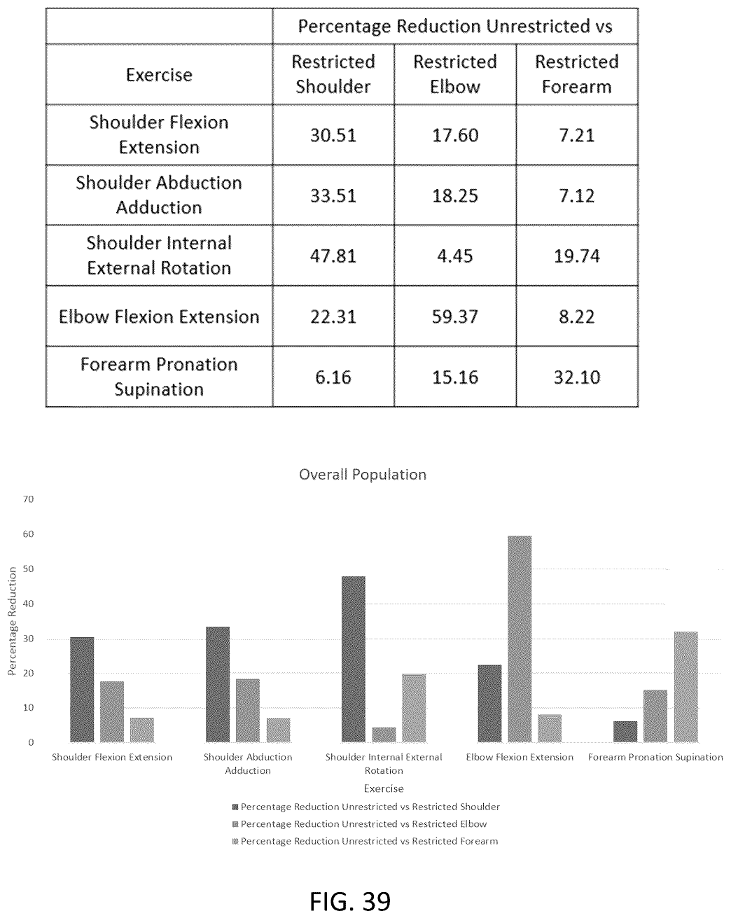

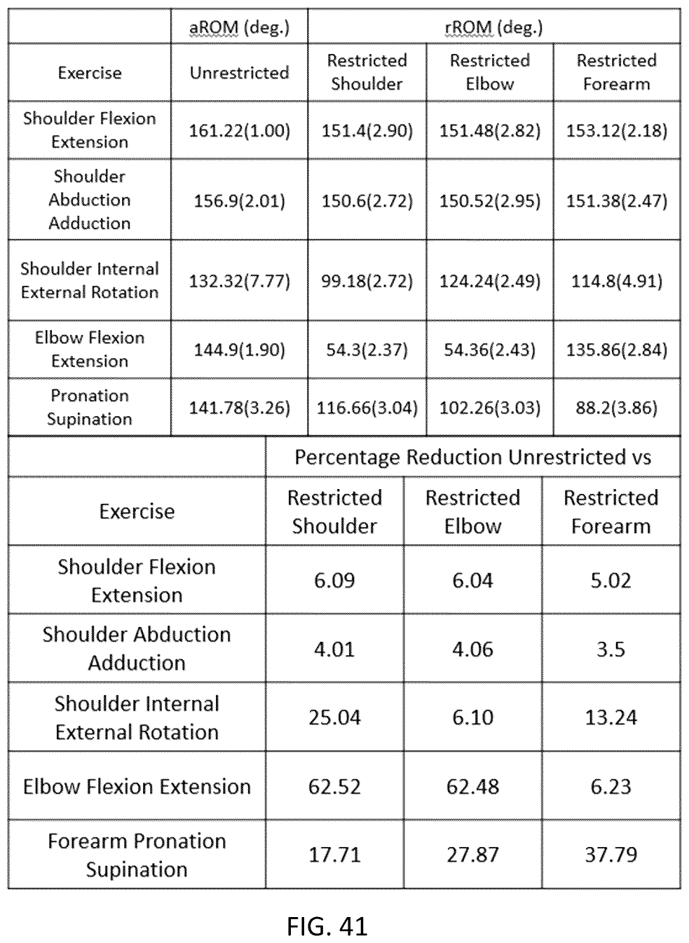

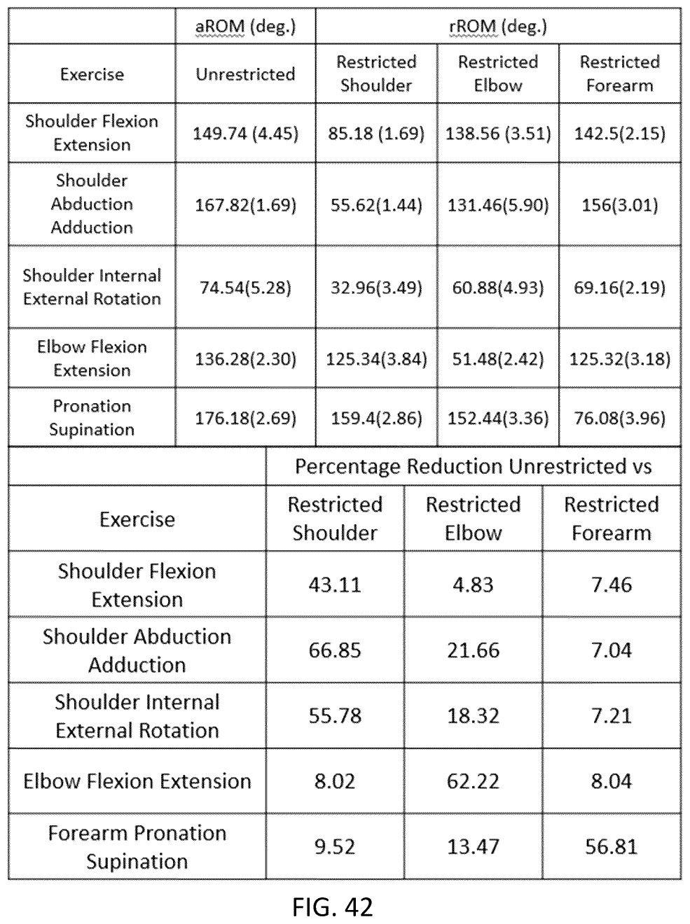

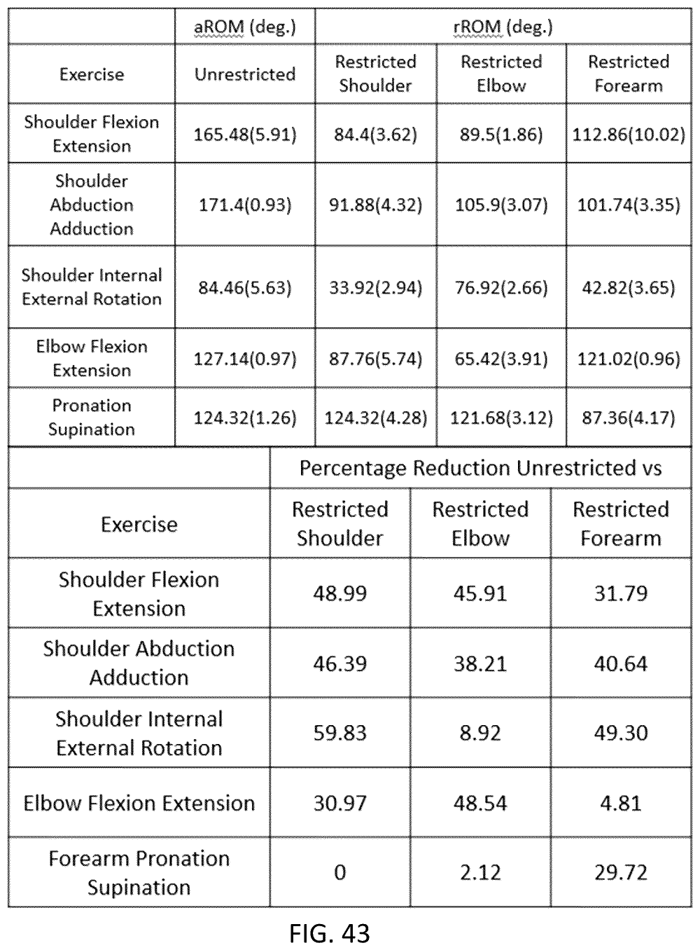

The WISE data was collected and analyzed (FIG. 38 through FIG. 43), which led to the following observations: (a) shoulder restriction reduced shoulder elevation, shoulder internal/external rotation, and elbow flexion extension; (b) elbow restriction reduced shoulder elevation, elbow flexion/extension, and forearm pronation/supination; and (c) forearm restriction reduced forearm pronation/supination. In the male data (FIG. 35), elbow restrictions were observed to have the smallest effect on shoulder internal-external rotation, but have almost equal effect on forearm movement as forearm restrictions. In the female data (FIG. 37), larger reductions in primary and secondary movements compared to males were observed, although the general pattern is similar. Forearm restrictions, however, had a larger effect on forearm movements than elbow movements. This difference distinguished females from males.

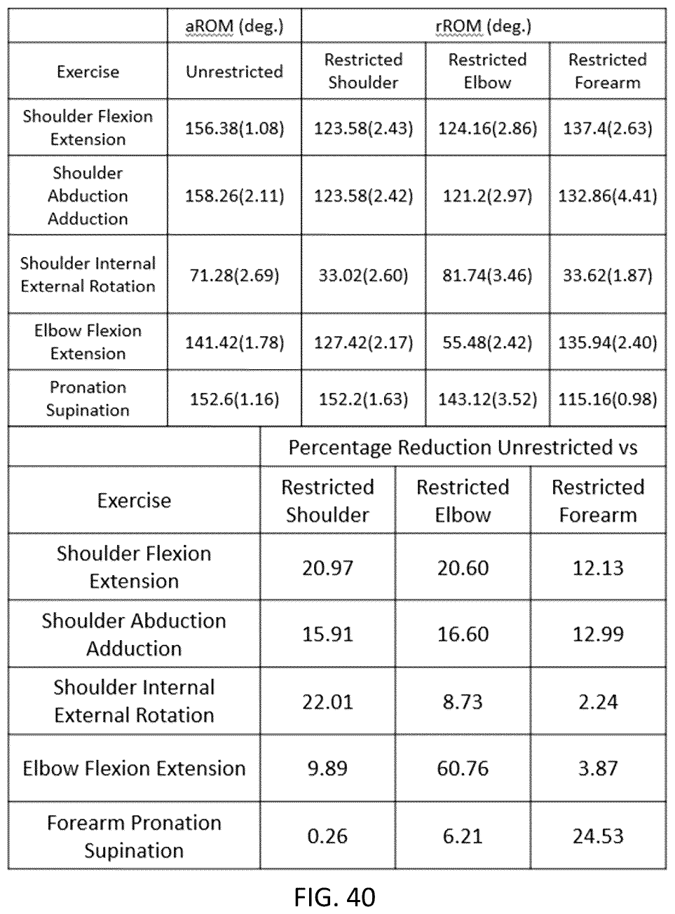

It is also possible to examine individual subject patterns of primary and secondary movement reductions over time before and after therapy and identify whether there is true improvement (i.e., the movement is restored) or if the improvement is due to compensation (i.e., movements other than the restricted joints increase). Some examples of individual patterns are shown in FIG. 40 through FIG. 43. For example, male subject 3 experienced greater percentage reduction in shoulder elevation for shoulder joint exercises than male subject 6, however restriction at shoulder affected elbow flexion and extension and restriction at elbow affected forearm pronation and supination for male subject 6 but not for male subject 3. In another example, restriction at shoulder affected elbow flexion extension and restriction at forearm affected shoulder flexion extension and shoulder abduction adduction for female subject 4 but not for female subject 2, while restriction at elbow affected forearm pronation and supination for female subject 2 but not for female subject 4.

The results show that restriction at the dominant joint reduced the range of motion for the corresponding joint to the maximum extent. Moreover, it shows that shoulder restriction impacted elbow flexion/extension and elbow restriction impacted shoulder elevation and forearm pronation/supination. A restriction at one joint may also lead to increased compensatory movements at other joints, which are difficult to differentiate clinically. By capturing movements at multiple joints simultaneously in real time and using algorithms based on the patterns of joint restriction in more than one joint, it is possible to identify (1) primary joint restrictions, (2) secondary joint restrictions, and (3) compensatory movements. Identifying the impact of joint restriction in this manner is possible only with the WISE system working as a whole unit.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A wearable inertial sensor system to detect upper extremity movement, comprising:
a plurality of inertial sensors configured to removably connect to a plurality of mounting devices; and
a computing system wirelessly and communicatively connected to the plurality of inertial sensors, further configured to provide a plurality of user interfaces for rehabilitative exergames;
wherein the wearable inertial sensor system is configured to provide a plurality of real-time joint angles based on the position and orientation of the plurality of inertial sensors.

2. The system of claim 1, wherein a plurality of joint angles are calculated in a joint angle coordinate system by the computing system based on quaternion data provided by the plurality of inertial sensors.

3. The system of claim 2, wherein the joint angels are calculated in real time and shown in an exergame user interface to facilitate rehabilitation and therapeutics.

4. The system of claim 1, wherein one of the plurality of inertial sensors is configured to be placed on a subject's left forearm.

5. The system of claim 1, wherein one of the plurality of inertial sensors is configured to be placed on a subject's left upper arm.

6. The system of claim 1, wherein one of the plurality of inertial sensors is configured to be placed on a subject's right forearm.

7. The system of claim 1, wherein one of the plurality of inertial sensors is configured to be placed on a subject's right upper arm.

8. The system of claim 1, wherein one of the plurality of inertial sensors is configured to be placed centrally on a subject's back.

9. The system of claim 1, further comprising a calibration device.

10. The system of claim 1, wherein one of the plurality of user interfaces is a sensor calibration user interface.

11. The system of claim 1, wherein one of the plurality of user interfaces is a sensor mounting user interface that facilitates the configuration of sensors for each individual.

12. The system of claim 1, wherein one of the plurality of user interfaces is a patient user interface with exergames to perform range of motion exercises.

13. The system of claim 1, wherein one of the plurality of user interfaces is a playback user interface with the ability for a clinician or therapist to view joint angles and assess subject performance.

14. The system of claim 1, wherein one of the plurality of user interfaces is an instructor user interface with the ability to develop new exercises by a clinician for a subject to perform.

15. The system of claim 1, further comprising a non-transitory computer-readable medium with instructions stored thereon, that when executed by a processor, performs the steps of:
calibrating the plurality of inertial sensors;
correcting for gravity based misalignment;
correcting for magnetic field based misalignment;
collecting inertial sensor data;
calculating relative quaternion positions between the inertial sensors;
converting the calculated quaternion positions to joint angles; and
displaying the joint angles to a user via a user interface.

16. The system of claim 15, wherein the joint angles are displayed to a user via the user interface in real-time.

17. A joint angle calculation method, comprising:
calibrating a plurality of inertial sensors;
correcting for gravity based misalignment;
correcting for magnetic field based misalignment;
collecting inertial sensor data;
calculating relative quaternion positions between the inertial sensors;
converting the calculated quaternion positions to joint angles; and
displaying the joint angles to a user via a user interface.

18. A non-transitory computer-readable medium for calculating joint angles for exergames, comprising:
a computer program code segment used to communicate with a plurality of inertial sensors;
a computer program code segment used to calibrate the plurality of inertial sensors;
a computer program code segment used to collect inertial sensor data;
a computer program code segment used to calculate relative quaternion positions between the inertial sensors;
a computer program code segment used to convert the calculated quaternion positions to joint angles; and
a computer program code segment used to present the joint angles on a exergame user interface in real time.

19. The computer-readable medium of claim 18, wherein the computer-readable medium includes instructions stored thereon, that when executed by a processor, performs the steps of:
calibrating the plurality of inertial sensors;
correcting for gravity based misalignment;
correcting for magnetic field based misalignment;
collecting inertial sensor data;
calculating relative quaternion positions between the inertial sensors;
converting the calculated quaternion positions to joint angles; and
displaying the joint angles to a user via a user interface.

20. A method of accurately placing inertial sensors on a subject, comprising the steps of:
placing a first inertial sensor on an upper body of the subject;
placing a second inertial sensor on one or both of the subject's left and right upper arms; and
placing a third inertial sensor on one or both of the subject's left and right forearms;
wherein a carrying angle of one or both of the subject's left and right arms is between about 8° and 20°, and wherein an internal-external rotation of one or both of the subject's left and right shoulder joints is within about 5° of a neutral pose.

21. The method of claim 20, wherein the first inertial sensor is positioned on a lower back of the subject.

22. The method of claim 20, wherein the second inertial sensor is positioned just proximal to one or both of the subject's elbow joint.

23. The method of claim 20, wherein the third inertial sensor is positioned just proximal to one or both of the subject's wrist joint.

24. A method of diagnosing an upper body mobility disease or disorder in a subject, comprising the steps of:
measuring a range of motion (ROM) in one or more joints of the subject;
measuring a deviation in the measured joint ROM from a baseline joint ROM; and
characterizing one or more diseases or disorders in the subject based on the measured deviations.

25. The method of claim 24, wherein the joint is selected from the group consisting of: the shoulder, the elbow, and the wrist.

26. The method of claim 24, wherein the joint ROM is selected from the group consisting of:
shoulder flexion, shoulder extension, shoulder abduction, shoulder adduction, shoulder internal rotation, shoulder external rotation, shoulder protraction, shoulder retraction, shoulder plane, shoulder elevation, elbow flexion, elbow extension, carrying angle, wrist pronation, wrist supination, wrist radial deviation, wrist ulnar deviation, wrist palmarflexion, wrist dorsiflexion, finger flexion, finger extension, finger abduction, finger adduction, thumb flexion, thumb extension, thumb opposition, thumb abduction, thumb adduction. The embodiment can be extended to include neck flexion, neck extension, neck rotation, next lateral bending, spine flexion, spine extension, spine lateral bending, spine rotation, hip flexion, hip extension, knee flexion, knee extension, ankle plantar flexion, ankle dorsiflexion, eversion, inversion, toe flexion, and toe extension.

27. The method of claim 24, wherein the disease or disorder is selected from the group consisting of: stroke, multiple sclerosis, spinal cord injury, nerve damage, rheumatism, arthritis, fracture, sprain, stiffness, weakness, impaired coordination, impaired proprioception, epicondylitis, tendonitis, and hypermobility.

28. The method of claim 24, wherein the deviation is an increase or decrease in joint ROM of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

29. The method of claim 24, wherein movement impairment is detected in joints having movement in a sagittal plane, a frontal plane, and/or a horizontal plane.

30. The method of claim 24, wherein the baseline joint ROM is derived from a population selected from the group consisting of: a global population, a regional group, an ethnic group, an age group, a gender group, a healthy subject, a subject having a disorder or disease, a subject at a stage of progression of a disorder or disease, and a subject at a stage of treatment of a disorder or disease.

31. The method of claim 24, wherein a derived joint ROM is used to predict a progression of a disease or disorder and a therapeutic intervention based on characteristic patterns of ROM deviation from baseline specific to the disease or disorder.

32. The method of claim 24, wherein the method further comprises a step of administering a treatment and a step of measuring changes in joint ROM after treatment.
